(19) United States
(12) Patent Application Publication
(10) Pub. No.: US 2007/0169219 A1
(43) Pub. Date: Jul. 19, 2007

(54) Nucleotide sequences and corresponding polypeptides conferring improved nitrogen use efficiency characteristics in plants

(76) Inventors: Greg Nadzan, Woodland Hills, CA (US); Richard Schneeberger, Carlsbad, CA (US); Kenneth Feldmann, Newbury Park, CA (US)

Correspondence Address: BIRCH STEWART KOLASCH & BIRCH, PO BOX 747, FALLS CHURCH, VA, 22040-0747, US

(21) Appl. No.: 11/654,357

(22) Filed: Jan. 16, 2007

Related U.S. Application Data
(60) Provisional application No. 60/778,568, filed on Mar. 1, 2006.
(60) Provisional application No. 60/758,831, filed on Jan. 13, 2006.

(51) Int. Cl. A01H 1/00; C07H 21/04; C12N 15/82; C12N 5/04

(52) U.S. Cl. 800/278; 435/468; 435/419; 536/23.6

(57) ABSTRACT

The present invention relates to isolated nucleic acid molecules and their corresponding encoded polypeptides able to confer the traits of improved nitrogen use efficiency in plants. The present invention further relates to the use of these nucleic acid molecules and polypeptides in making transgenic plants, plant cells, plant materials or seeds of a plant having improved nitrogen use efficiency that leads to improvement in plant size, vegetative growth, growth rate, seedling vigor and/or biomass that are altered with respect to wild type plants grown under normal and/or abnormal nitrogen conditions.

### CROSS-REFERENCE TO RELATED APPLICATIONS

This non-provisional application claims priority under 35 U.S.C. § 119(e) on U.S. Provisional application Nos. 60/778,568 filed on Mar. 1, 2006, and 60/758,831 filed on Jan. 13, 2006, the entire contents of which are hereby incorporated by reference.

This application contains a CDR, the entire contents of which are hereby incorporated by reference. The CDR contains the following files:

| File Name | Create Date | File Size |
|---|---|---|
| Jan. 11, 2007 2750-1668PUS2 Revised | Jan. 16, 2007 | 460 KB |
| Sequence_Listing.txt | | |

### FIELD OF THE INVENTION

The present invention relates to isolated nucleic acid molecules and their corresponding encoded polypeptides able to improve nitrogen use efficiency in plants. The present invention further relates to using the nucleic acid molecules and polypeptides to make transgenic plants, plant cells, plant materials or seeds of a plant having improved nitrogen use efficiency as compared to wild-type plants grown under similar normal and/or abnormal nitrogen conditions. This application claims priority to U.S. application No: 60/778,568, filed Mar. 1, 2006 and U.S. application No: 60/758,831, filed Jan. 13, 2006.

### BACKGROUND OF THE INVENTION

Plants specifically improved for agriculture, horticulture, biomass conversion, and other industries (e.g. paper industry, plants as production factories for proteins or other compounds) can be obtained using molecular technologies. As an example, great agronomic value can result from enhancing plant growth under low nitrogen conditions.

Nitrogen is most frequently the rate limiting mineral nutrient for crop production and all field crops have a fundamental dependence on exogenous nitrogen sources. Nitrogenous fertilizer, which is usually supplied as ammonium nitrate, potassium nitrate or urea, typically accounts for 40% of the costs associated with crops in intensive agriculture, such as corn and wheat. Increased efficiency of nitrogen use by plants enables the production of higher yields with existing fertilizer inputs, enables existing crop yields to be obtained with lower fertilizer input or enables better yields from soils of poorer quality (Good et al. (2004) Trends Plant Sci. 9:57-605). Higher amounts of proteins in the crops can also be produced more cost-effectively.

Interestingly, high concentrations of nitrogen are known to be toxic to plants, especially at the seedling stage (Brenner and Krogmeier (1989) PNAS 86:8185-8188). Here, abnormally high nitrogen concentrations create toxic nitrogen effects (“burning”) and/or leads to the inhibition of germination, reducing yield as a consequence. This is a particular problem during the application of urea and other ammonium based fertilizers since segments of a planting field can vary widely in the available nitrogen present and high ammonium levels are toxic to plants. Most crop plants are severely damaged by high nitrogen conditions, so yield can be significantly reduced.

Plants have a number of means to cope with nitrogen nutrient deficiencies, such as poor nitrogen availability. One important mechanism senses nitrogen availability in the soil and responds accordingly by modulating gene expression while a second mechanism is to sequester or store nitrogen in times of abundance to be used later. Yet the particulars of these mechanisms and how they interact to govern nitrogen use efficiency in a competitive environment (i.e. low and/or high nitrogen) remain largely unanswered.

The nitrogen sensing mechanism relies on regulated gene expression and enables rapid physiological and metabolic responses to changes in the supply of inorganic nitrogen in the soil by adjusting nitrogen uptake, reduction, partitioning, remobilization and transport in response to changing environmental conditions. Nitrate acts as a signal to initiate a number of responses that serve to reprogram plant metabolism, physiology and development (Redinbaugh et al. (1991) Physiol. Plant. 82, 640-650.; Forde (2002) Annual Review of Plant Biology 53, 203-224). Nitrogen-inducible gene expression has been characterized for a number of genes in some detail. These include nitrate reductase, nitrite reductase, 6-phosphoglucante dehydrogenase, and nitrate and ammonium transporters (Redinbaugh et al. (1991) Physiol. Plant. 82, 640-650; Huber et al. (1994) Plant Physiol 106, 1667-1674; Hwang et al. (1997) Plant Physiol. 113, 853-862; Redinbaugh et al. (1998) Plant Science 134, 129-140; Gazzarrini et al. (1999) Plant Cell 11, 937-948; Glass et al. (2002) J. Exp. Bot. 53, 855-864; Okamoto et al. (2003) Plant Cell Physiol. 44, 304-317).

Investigations into the cis acting control elements and DNA binding factors involved in nitrate regulated gene expression have focused on the nitrate reductase genes from tobacco and spinach, and have identified several putative regulatory elements ( Rastogi et al. (1993) Plant J 4, 317-326; Lin et al. (1994) Plant Physiol. 106, 477-484; Hwang et al. (1997) Plant Physiol. 113, 853-862). Transcriptional profiling of nitrate-regulated gene expression has extended knowledge of genes and processes regulated by nitrate availability and also identified a number of genes with distinct spatial and temporal patterns of expression (Ceres unpublished; Wang et al. (2000) Plant Cell 12, 1491-1510; Wang et al. (2003) Plant Physiol. 132, 556-567).

Inefficiencies in nitrogen use efficiency (NUE) may be overcome through the use of nitrogen regulated gene expression to modify the response of rate limiting enzymes and metabolic pathways that occur in response to changes in nitrogen availability. General reviews of these pathways and processes can be found in: Derlot et al. (2001) Amino Acid Transport. In Plant Nitrogen (eds. Lea and Morot-Gaudry), pp. 167-212. Springer-Verlag, Berlin, Heidelberg; Glass et al. (2002) J. Exp. Bot. 53: 855-864; Krapp et al. (2002) Nitrogen and Signaling. In Photosynthetic Nitrogen Assimilation and Associated Carbon Respiratory Metabolism (eds. Foyer and Noctor), pp. 205-225. Kluwer Academic Publisher, Dordrecht, The Netherlands; and Touraine et al. (2001) Nitrate uptake and its regulation. In Plant Nitrogen (eds. Lea and Morot-Gaudry), pp. 1-36. Springer-Verlag, Berlin, Heidelberg. Overcoming the rate limiting steps in nitrogen assimilation, transport and metabolism has the effect of increasing the yield, reducing the nitrogen content and reducing the protein content of plants grown under nitrogen limiting conditions.

The availability and sustainability of a stream of food and feed for people and domesticated animals has been a high priority throughout the history of human civilization and lies at the origin of agriculture. Specialists and researchers in the fields of agronomy science, agriculture, crop science, horticulture and forest science are even today constantly striving to find and produce plants with an increased growth potential to feed an increasing world population and to guarantee a supply of reproducible raw materials. The robust level of research in these fields of science indicates the level of importance leaders in every geographic environment and climate around the world place on providing sustainable sources of food, feed and energy.

Manipulation of crop performance has been accomplished conventionally for centuries through plant breeding. The breeding process is, however, both time-consuming and labor-intensive. Furthermore, appropriate breeding programs must be specially designed for each relevant plant species.

On the other hand, great progress has been made in using molecular genetics approaches to manipulate plants to provide better crops. Through introduction and expression of recombinant nucleic acid molecules in plants, researchers are now poised to provide the community with plant species tailored to grow more efficiently and produce more product despite unique geographic and/or climatic environments. These new approaches have the additional advantage of not being limited to one plant species, but instead being applicable to multiple different plant species (Zhang et al. (2004) Plant Physiol. 135:615; Zhang et al (2001) Pro. Natl. Acad. Sci. USA 98:12832).

Despite this progress, today there continues to be a great need for generally applicable processes that improve forest or agricultural plant growth to suit particular needs depending on specific environmental conditions. To this end, the present invention is directed to improving nitrogen use efficiency to maximize plant growth in various crops depending on the particular environment in which the crop must grow, characterized by expression of recombinant DNA molecules in plants. These molecules may be from the plant itself, and simply expressed at a higher or lower level, or the molecules may be from different plant species.

### SUMMARY OF THE INVENTION

The present invention, therefore, relates to isolated nucleic acid molecules and polypeptides and their use in making transgenic plants, plant cells, plant materials or seeds of plants having improved NUE when compared to wild-type plants grown under similar or identical normal and/or abnormal nitrogen conditions.

The present invention also relates to processes for increasing the growth potential in plants due to NUE, recombinant nucleic acid molecules and polypeptides used for these processes, as well as to plants with an increased growth potential due to improved NUE. The phrase “increasing growth potential” refers to continued growth under low or high nitrogen conditions, better soil recovery after exposure to low or high nitrogen conditions and increased tolerance to varying nitrogen conditions. Such an increase in growth potential preferably results from an increase in NUE.

Unless otherwise defined, all technical and scientific terms used herein have the same meaning as commonly understood by one of ordinary skill in the art to which this invention belongs.

### BRIEF DESCRIPTION OF THE FIGURES

FIG. 1. Amino acid sequence alignment of homologues of Lead 82 (ME02507), SEQ ID NO: 81. Conserved regions are enclosed in a box. A consensus sequence is shown below the alignment.

FIG. 2. Amino acid sequence alignment of homologues of Lead 92 (ME08309), SEQ ID NO: 107. Conserved regions are enclosed in a box. A consensus sequence is shown below the alignment.

FIG. 3. Amino acid sequence alignment of homologues of ME03926, SEQ ID NO: 201. Conserved regions are enclosed in a box. A consensus sequence is shown below the alignment.

FIG. 4. Amino acid sequence alignment of homologues of Lead ME07344, SEQ ID NO: 140. Conserved regions are enclosed in a box. A consensus sequence is shown below the alignment.

FIG. 5. Amino acid sequence alignment of homologues of Lead 93 (ME10822), SEQ ID NO: 114. Conserved regions are enclosed in a box. A consensus sequence in shown below the alignment.

### DETAILED DESCRIPTION OF THE INVENTION

1. The Invention

The invention of the present application may be described by, but not necessarily limited to, the following exemplary embodiments.

The present invention discloses novel isolated nucleic acid molecules, nucleic acid molecules that interfere with these nucleic acid molecules, nucleic acid molecules that hybridize to these nucleic acid molecules, and isolated nucleic acid molecules that encode the same protein due to the degeneracy of the DNA code. Additional embodiments of the present application further include the polypeptides encoded by the isolated nucleic acid molecules of the present invention.

More particularly, the nucleic acid molecules of the present invention comprise: (a) a nucleotide sequence encoding an amino acid sequence that is at least 85% identical to any one of Leads 82, 92, 93, 98, ME07344, ME05213, ME02730 and ME24939 corresponding to SEQ ID NO: 81, 105, 107, 114, 116, 201, 140, 84, 112 and 200, respectively, (b) a nucleotide sequence that is complementary to any one of the nucleotide sequences according to (a), (c) a nucleotide sequence according to any one of SEQ ID Nos. NO: 80, 104, 106, 113, 115, 127, 139, 202, 203 and 204, (d) a nucleotide sequence able to interfere with any one of the nucleotide sequences according to (a), (e) a nucleotide sequence able to form a hybridized nucleic acid duplex with the nucleic acid according to any one of paragraphs (a)-(e) at a temperature from about 40° C. to about 48° C. below a melting temperature of the hybridized nucleic acid duplex, and (f) a nucleotide sequence encoding any one of amino acid sequences of Leads 82, 85, 92, 93, 98, 112, ME07344, ME05213, ME02730 and ME24939, corresponding to SEQ ID NOS: 81, 105, 107, 114, 116, 201, 140, 84, 112 and 200, respectively.

Additional embodiments of the present invention include those polypeptide and nucleic acid molecule sequences disclosed in SEQ ID NOS: 80, 81, 104, 105, 106, 107, 113, 114, 115, 116, 127, 128, 139, 140, 84, 112 and 200-204.

The present invention further embodies a vector comprising a first nucleic acid having a nucleotide sequence encoding a plant transcription and/or translation signal, and a second nucleic acid having a nucleotide sequence according to the isolated nucleic acid molecules of the present invention. More particularly, the first and second nucleic acids may be operably linked. Even more particularly, the second nucleic acid may be endogenous to a first organism, and any other nucleic acid in the vector may be endogenous to a second organism. Most particularly, the first and second organisms may be different species.

In a further embodiment of the present invention, a host cell may comprise an isolated nucleic acid molecule according to the present invention. More particularly, the isolated nucleic acid molecule of the present invention found in the host cell of the present invention may be endogenous to a first organism and may be flanked by nucleotide sequences endogenous to a second organism. Further, the first and second organisms may be different species. Even more particularly, the host cell of the present invention may comprise a vector according to the present invention, which itself comprises nucleic acid molecules according to those of the present invention.

In another embodiment of the present invention, the isolated polypeptides of the present invention may additionally comprise amino acid sequences that are at least 85% identical to any one of Leads 82, 85, 92, 93, 98, 112, ME07344, ME05213, ME02730 and ME24939, corresponding to SEQ ID NOS: 81, 105, 107, 114, 116, 201, 140, 84, 112 and 200, respectively.

Other embodiments of the present invention include methods of introducing an isolated nucleic acid of the present invention into a host cell. More particularly, an isolated nucleic acid molecule of the present invention may be contacted to a host cell under conditions allowing transport of the isolated nucleic acid into the host cell. Even more particularly, a vector as described in a previous embodiment of the present invention, may be introduced into a host cell by the same method.

Methods of detection are also available as embodiments of the present invention. Particularly, methods for detecting a nucleic acid molecule according to the present invention in a sample. More particularly, the isolated nucleic acid molecule according to the present invention may be contacted with a sample under conditions that permit a comparison of the nucleotide sequence of the isolated nucleic acid molecule with a nucleotide sequence of nucleic acid in the sample. The results of such an analysis may then be considered to determine whether the isolated nucleic acid molecule of the present invention is detectable and therefore present within the sample.

A further embodiment of the present invention comprises a plant, plant cell, plant material or seeds of plants comprising an isolated nucleic acid molecule and/or vector of the present invention. More particularly, the isolated nucleic acid molecule of the present invention may be exogenous to the plant, plant cell, plant material or seed of a plant.

A further embodiment of the present invention includes a plant regenerated from a plant cell or seed according to the present invention. More particularly, the plant, or plants derived from the plant, plant cell, plant material or seeds of a plant of the present invention preferably has improved NUE, increased size (in whole or in part), increased vegetative growth, and/or increased biomass (sometimes hereinafter collectively referred to as increased biomass) characteristics as compared to a wild-type plant cultivated under identical normal and/or abnormal nitrogen conditions. Furthermore, the transgenic plant may comprise a first isolated nucleic acid molecule of the present invention, which encodes a protein involved in modulating NUE, growth and phenotype characteristics, and a second isolated nucleic acid molecule which encodes a promoter capable of driving expression in plants, wherein the growth and phenotype modulating component and the promoter are operably linked. More preferably, the first isolated nucleic acid may be misexpressed in the transgenic plant of the present invention, and the transgenic plant exhibits modulated characteristics as compared to a progenitor plant devoid of the polynucleotide, when the transgenic plant and the progenitor plant are cultivated under identical normal and/or abnormal nitrogen environmental conditions. In another embodiment of the present invention the modulated NUE, growth and phenotype characteristics may be due to the inactivation of a particular sequence, using for example an interfering RNA.

A further embodiment consists of a plant, plant cell, plant material or seed of a plant according to the present invention which comprises an isolated nucleic acid molecule of the present invention, wherein the plant, or plants derived from the plant, plant cell, plant material or seed of a plant, has the modulated NUE, growth and phenotype characteristics as compared to a wild-type plant cultivated under identical normal and/or abnormal nitrogen conditions.

The polynucleotide conferring improved NUE, biomass or vigor may be mis-expressed in the transgenic plant of the present invention, and the transgenic plant exhibits an increased NUE, biomass or vigor as compared to a progenitor plant devoid of the polynucleotide, when the transgenic plant and the progenitor plant are cultivated under identical normal and/or abnormal nitrogen environmental conditions. In another embodiment of the present invention improved NUE, biomass or vigor phenotype exhibited under normal and/or abnormal nitrogen environmental conditions may be due to the inactivation of a particular sequence, using for example an interfering RNA.

Another embodiment consists of a plant, plant cell, plant material or seed of a plant according to the present invention which comprises an isolated nucleic acid molecule of the present invention, wherein the plant, or plants derived from the plant, plant cell, plant material or seed of a plant, has increased NUE, biomass or vigor as compared to a wild-type plant cultivated under identical normal and/or abnormal nitrogen conditions.

Another embodiment of the present invention includes methods of enhancing NUE, biomass or vigor in plants. More particularly, these methods comprise transforming a plant with an isolated nucleic acid molecule according to the present invention. Preferably, the method is a method of enhancing NUE, biomass or vigor in the transformed plant, whereby the plant is transformed with a nucleic acid molecule encoding the polypeptide of the present invention.

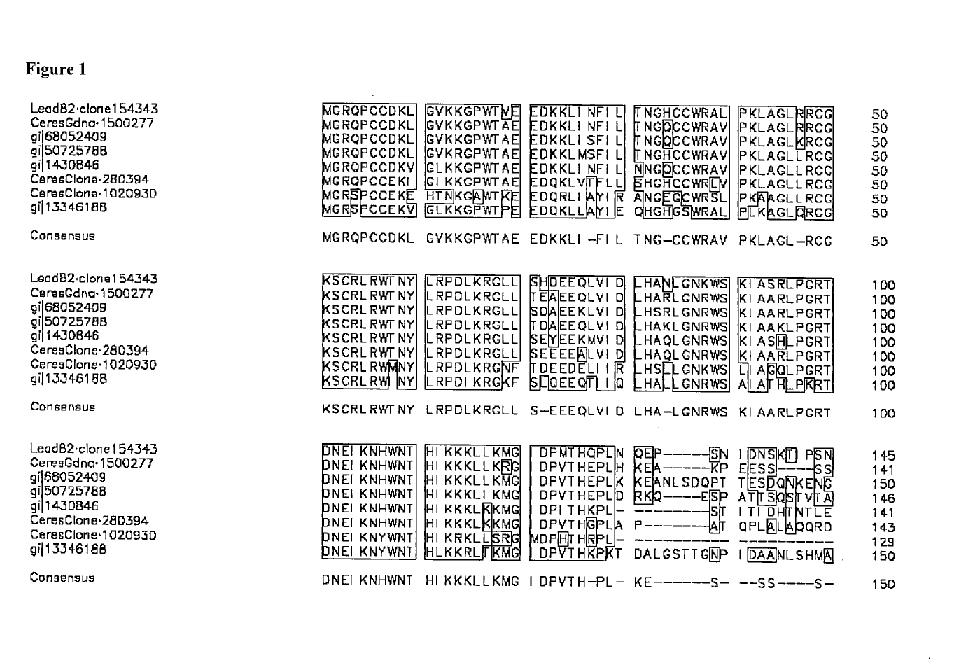

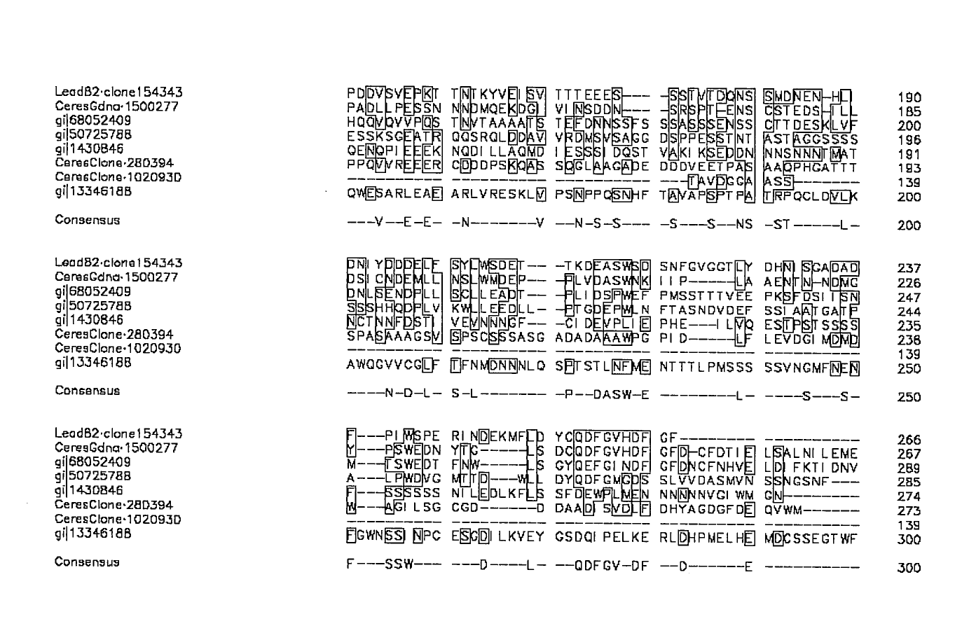

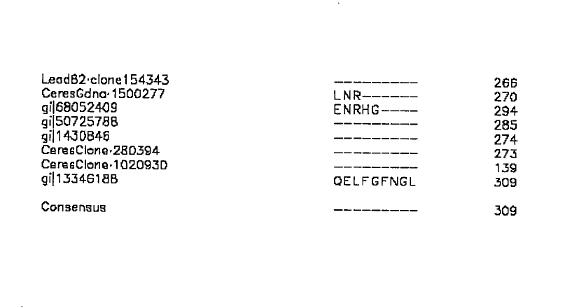

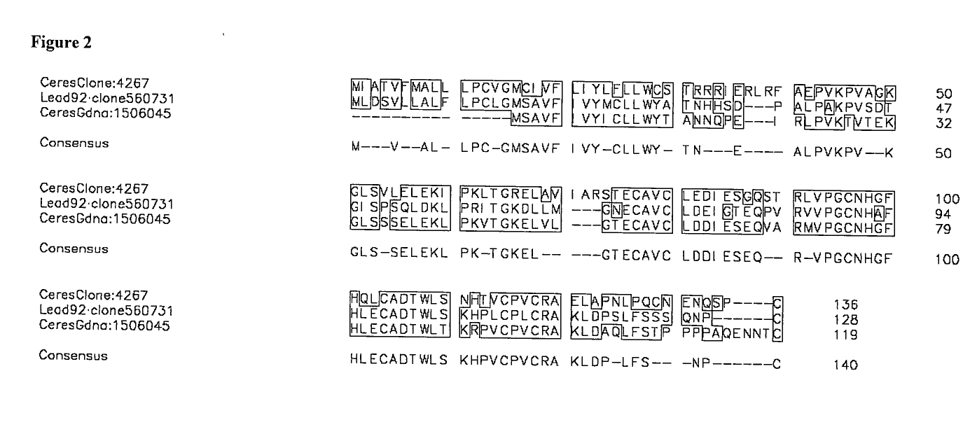

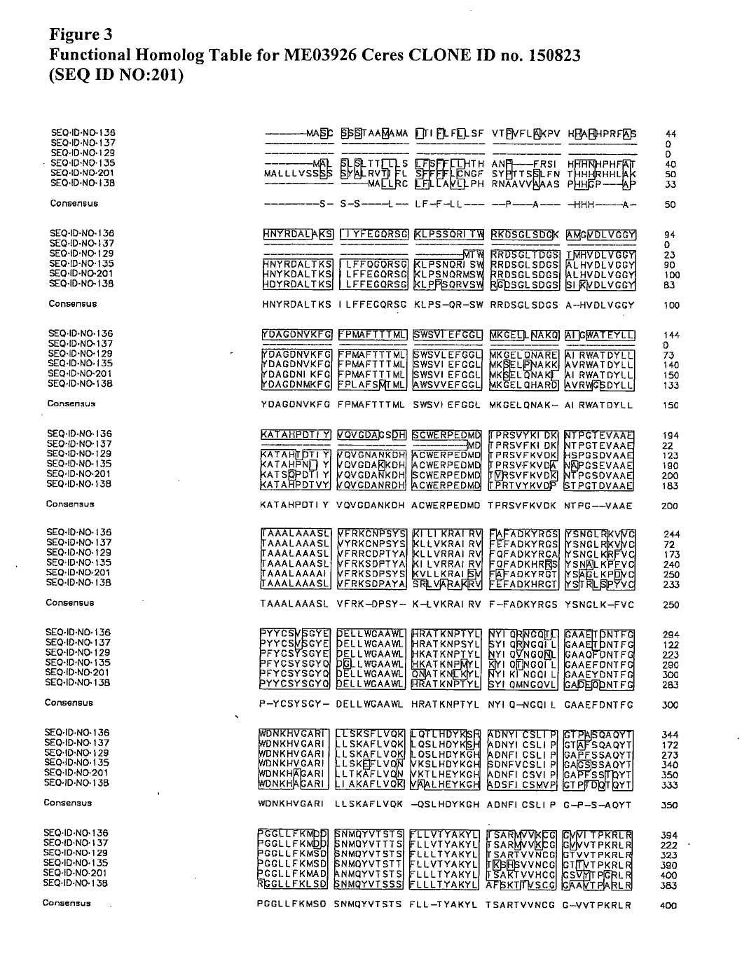

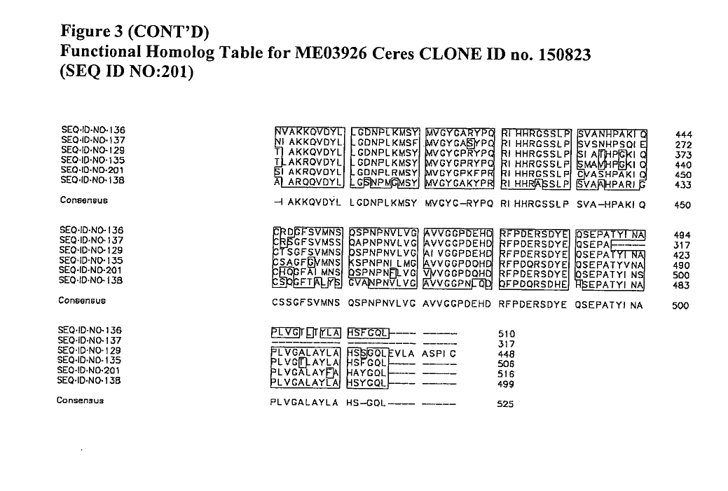

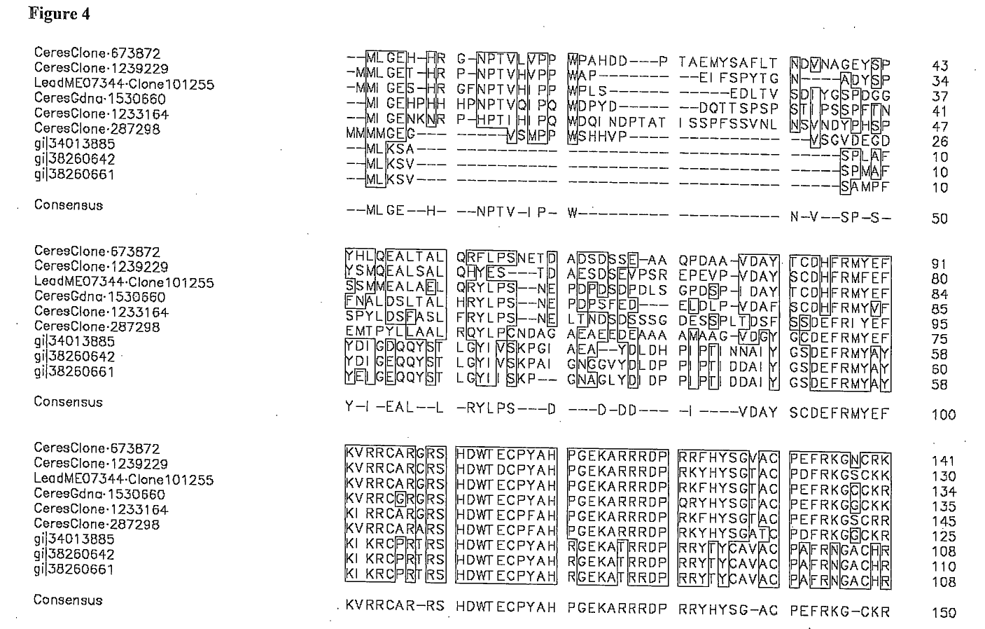

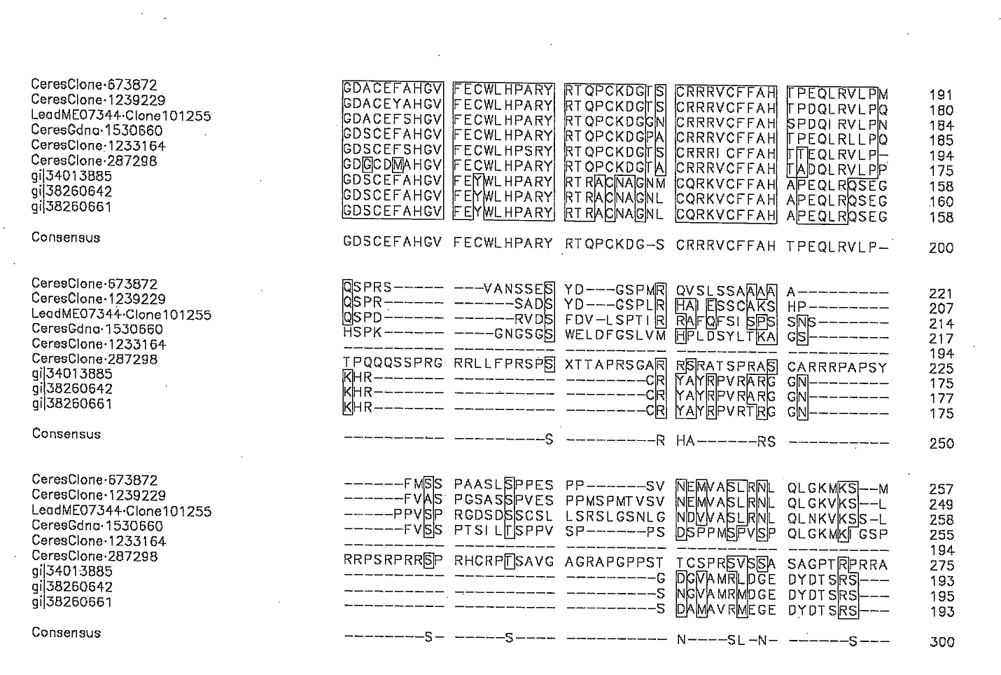

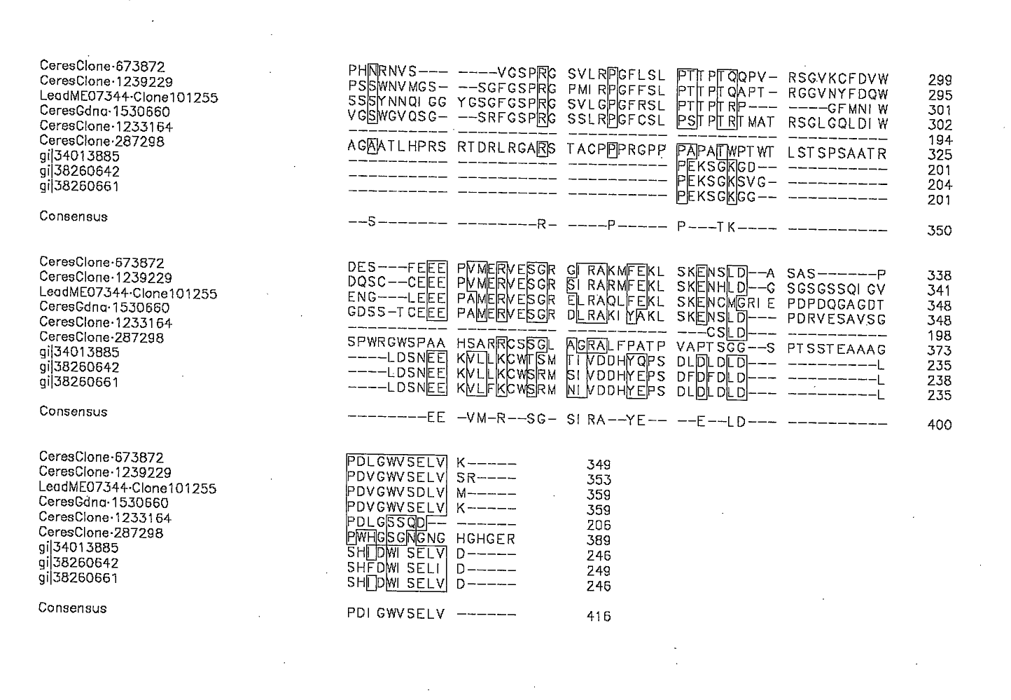

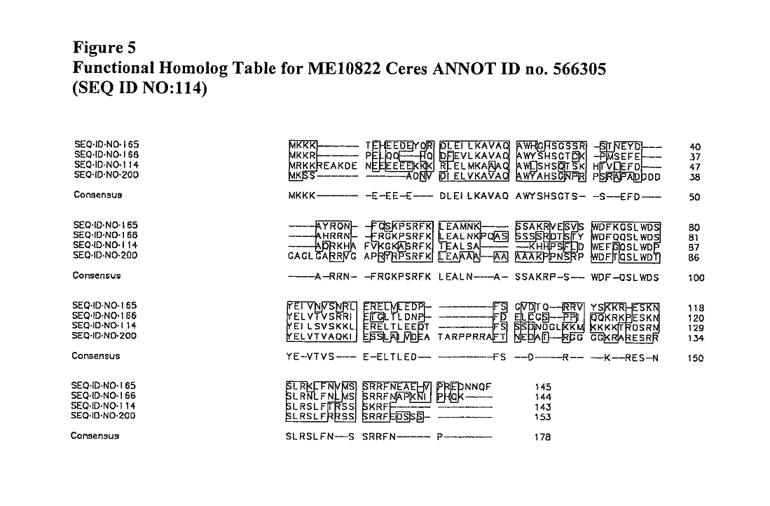

Polypeptides of the present invention include consensus sequences. The consensus sequences are those as shown in FIGS. 1-5.

2. Definitions

The following terms are utilized throughout this application:

Abnormal Nitrogen Conditions: Soil nitrogen levels can vary by 10 orders of magnitude, thus plant species vary in their capacity to tolerate particular nitrogen conditions. Nitrogen-sensitive plant species, including many agronomically important species, can be injured by nitrogen conditions that are either low or high compared to the range of nitrogen needed for normal growth. At nitrogen conditions above or below the range needed for normal growth, most plant species will be damaged or suffer reduced growth potential. Thus, “abnormal nitrogen conditions” can be defined as the nitrogen concentration at which a given plant species will be adversely affected as evidenced by symptoms such as decreased chlorophyll (for example, measured by chlorophyll a/b absorbance) decreased photosynthesis (for example, measured by CO2 fixation), membrane damage (for example, measured by electrolyte leakage), chlorosis (for example, via visual inspection), loss of biomass or seed yield. Since plant species vary in their capacity to tolerate abnormal nitrogen conditions, the precise environmental conditions that cause nitrogen stress can not be generalized. However, nitrogen tolerant plants are characterized by their ability to retain their normal appearance or recover quickly from abnormal nitrogen conditions. Such nitrogen tolerant plants produce higher biomass and yield than plants that are not nitrogen tolerant. Differences in physical appearance, recovery and yield can be quantified and statistically analyzed using well known measurement and analysis methods.

Plant seedlings vary considerably in their ability to grow under abnormal nitrogen conditions. Generally, seedlings of many plant species will not grow well at nitrogen concentration less than about 1 ppm or greater than about 750 ppm. High concentrations of ammoniac nitrogen are also inhibitory to seed germination and seedling growth and can occur when ammonium based fertilizer is used (Brenner and Krogmeier (1989) PNAS 86:8185-8188).

Once seeds have imbibed water they become very susceptible to disease, water and chemical damage. Seeds and seedlings that are tolerant to nitrogen stress during germination can survive for relatively long periods under which the nitrogen concentration is too high or too low for normal growth. Since plant species vary in their capacity to tolerate abnormal nitrogen conditions during germination, the precise environmental conditions that cause nitrogen stress during germination can not be generalized. However, seeds and seedlings that are nitrogen tolerant during germination are characterized by their ability to remain viable or recover quickly from low or high nitrogen conditions. Such nitrogen tolerant plants germinate, become established, grow more quickly and ultimately produce more biomass and yield than plants that are not nitrogen tolerant. Differences in germination rate, appearance, recovery and yield can be quantified and statistically analyzed using well known measurement and analysis methods.

Functionally Comparable Proteins or Functional Homologs: This phrase describes a set of proteins that perform similar functions within an organism. By definition, perturbation of an individual protein within that set (through misexpression or mutation, for example) is expected to confer a similar phenotype as compared to perturbation of any other individual protein. Such proteins typically share sequence similarity resulting in similar biochemical activity. Within this definition, homologs, orthologs and paralogs are considered to be functionally comparable.

Functionally comparable proteins will give rise to the same characteristic to a similar, but not necessarily the same, degree. Typically, comparable proteins give the same characteristics where the quantitative measurement due to one of the comparables is at least 20% of the other; more typically, between 30 to 40%; even more typically, between 50-60%; even more typically between 70 to 80%; even more typically between 90 to 100% of the other.

Heterologous sequences: “Heterologous sequences” are those that are not operatively linked or are not contiguous to each other in nature. For example, a promoter from corn is considered heterologous to an Arabidopsis coding region sequence. Also, a promoter from a gene encoding a growth factor from corn is considered heterologous to a sequence encoding the corn receptor for the growth factor. Regulatory element sequences, such as UTRs or 3′ end termination sequences that do not originate in nature from the same gene as the coding sequence, are considered heterologous to said coding sequence. Elements operatively linked in nature and contiguous to each other are not heterologous to each other. On the other hand, these same elements remain operatively linked but become heterologous if other filler sequence is placed between them. Thus, the promoter and coding sequences of a corn gene expressing an amino acid transporter are not heterologous to each other, but the promoter and coding sequence of a corn gene operatively linked in a novel manner are heterologous.

High Nitrogen Conditions: This phrase refers to total nitrogen concentrations that will result in growth retardation or tissue damage due to ionic or osmotioc stress. Growth medium concentrations of nitrogen that will lead to nitrogen stress can not be generalized. However, nitrogen concentrations that reduce germination rate by more than 20%, 25%. 30%, 35%, 40%, 45% or 50% are considered to be high and in excess.

Low Nitrogen Conditions: The phrase “low nitrogen conditions” refers to nitrogen concentrations which lead to nitrogen deficiency symptoms such as pale green leaf color, chlorosis and reduced growth and vigor. These concentrations of nitrogen are generally less than 10 ppm nitrate in a soil nitrate test. Typically, low nitrogen conditions lead to a reduction of at least 20%, 25%, 30%, 35%, 40%, 45%, 50%, 60%, 70%, 80% or 90growth and/or vigor.

Misexpression: The term “misexpression” refers to an increase or a decrease in the transcription of a coding region into a complementary RNA sequence as compared to the wild-type. This term also encompasses expression and/or translation of a gene or coding region or inhibition of such transcription and/or translation for a different time period as compared to the wild-type and/or from a non-natural location within the plant genome, including a gene or coding region from a different plant species or from a non-plant organism.

Nitrogen Use Efficiency: The efficiency with which plants utilize inorganic nitrogen to produce biomass and seeds is termed Nitrogen Use Efficiency (NUE). A number of different methods for measuring NUE, and components of NUE, are routinely used by scientists. NUE is usually measured as the amount of biomass or seed yield produced per unit of nitrogen applied to the soil. NUE can also be represented as the product of two factors, uptake efficiency and utilization efficiency. Nitrogen uptake efficiency measures the efficiency with which a plant removes nitrogen from the soil while utilization efficiency measures the yield obtained per unit of nutrient absorbed by a plant. A number of different biological processes are involved in defining a particular plant's NUE and can independently affect processes involved in uptake efficiency and utilization efficiency. Many of these processes are genetically determined and can be improved by genetic or biotechnologic manipulation of the genes responsible for determining these traits.

Normal Nitrogen Conditions: Plant species vary in their capacity to tolerate particular nitrogen conditions. Nitrogen-sensitive plant species, including many agronomically important species, can be injured by nitrogen conditions that are either low or high compared to the range of nitrogen needed for normal growth. At nitrogen conditions above or below the range needed for normal growth, most plant species will be damaged or suffer reduced growth potential. Thus, “normal nitrogen conditions” can be defined as the nitrogen concentration at which a given plant species will grow without damage. Since plant species vary in their capacity to tolerate nitrogen conditions, the precise environmental conditions that provide normal nitrogen conditions can not be generalized. However, the normal growth exhibited by nitrogen intolerant plants is characterized by the inability to retain a normal appearance or to recover quickly from abnormal nitrogen conditions. Such nitrogen intolerant plants produce lower biomass and yield less than plants that are nitrogen tolerant. Differences in physical appearance, recovery and yield can be quantified and statistically analyzed using well known measurement and analysis methods.

Plant seedlings vary considerably in their ability to grow under abnormal nitrogen conditions. Generally, seedlings of many plant species will not grow well at nitrogen concentration less than about 1 ppm or greater than about 750 ppm. High concentrations of ammoniac nitrogen are also inhibitory to seed germination and seedling growth and can occur when ammonium based fertilizer is used (Brenner and Krogmeier (1989) PNAS 86:8185-8188).

Once seeds have imbibed water they become very susceptible to disease, water and chemical damage. Seeds and seedlings that are tolerant to nitrogen stress during germination can survive for relatively long periods under which the nitrogen concentration is too high or too low for normal growth. Since plant species vary in their capacity to tolerate nitrogen conditions during germination, the precise environmental conditions that cause nitrogen stress during germination can not be generalized. However, the normal growth associated with nitrogen intolerant seeds is characterized by the inability to remain viable or recover quickly from low or high nitrogen conditions. Such nitrogen intolerant seeds do not germinate, do not become established, grow more slowly, if at all, and ultimately die faster or produce less biomass and yield than seeds that are nitrogen tolerant. Differences in germination rate, appearance, recovery and yield can be quantified and statistically analyzed using well known measurement and analysis methods.

Percentage of sequence identity: The term “percent sequence identity” refers to the degree of identity between any given query sequence, e.g. SEQ ID NO: 102, and a subject sequence. A subject sequence typically has a length that is from about 80 percent to 200 percent of the length of the query sequence, e.g., 82, 85, 87, 89, 90, 93, 95, 97, 99, 100, 105, 110, 115, or 120, 130, 140, 150, 160, 170, 180, 190 or 200 percent of the length of the query sequence. A percent identity for any subject nucleic acid or polypeptide relative to a query nucleic acid or polypeptide can be determined as follows. A query sequence (e.g. a nucleic acid or amino acid sequence) is aligned to one or more subject nucleic acid or amino acid sequences using the computer program ClustalW (version 1.83, default parameters), which allows alignments of nucleic acid or protein sequences to be carried out across their entire length (global alignment). Chenna et al. (2003) Nucleic Acids Res. 31 (13):3497-500.

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The ClustalW output is a sequence alignment that reflects the relationship between sequences. ClustalW can be run, for example, at the Baylor College of Medicine Search Launcher website and at the European Bioinformatics Institute website on the World Wide Web (ebi.ac.uk/clustalw).

To determine a percent identity of a subject or nucleic acid or amino acid sequence to a query sequence, the sequences are aligned using Clustal W, the number of identical matches in the alignment is divided by the query length, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

Photosynthetic efficiency: photosynthetic efficiency, or electron transport via photosystem II, is estimated by the relationship between Fm, the maximum fluorescence signal and the variable fluorescence, Fv. Here, a reduction in the optimum quantum. yield (Fv/Fm) indicates stress and can be used to monitor the performance of transgenic plants compared to non-transgenic plants under low nitrogen conditions.

Regulatory Regions: The term “regulatory region” refers to nucleotide sequences that, when operably linked to a sequence, influence transcription initiation or translation initiation or transcription termination of said sequence and the rate of said processes, and/or stability and/or mobility of a transcription or translation product. As used herein, the term “operably linked” refers to positioning of a regulatory region and said sequence to enable said influence. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Regulatory regions can be classified in two categories, promoters and other regulatory regions.

Seedling area: The total leaf area of a young plant about 2 weeks old.

Seedling vigor or vigor: As used herein, “seedling vigor” or “vigor” refers to the plant characteristic whereby the plant emerges from soil faster, has an increased germination rate (i.e., germinates faster), has faster and larger seedling or adult growth and/or germinates faster when grown under similar conditions as compared to the wild type or control under similar conditions. Seedling vigor has often been defined to comprise the seed properties that determine “the potential for rapid, uniform emergence and development of normal seedlings under a wide range of field conditions”.

Stringency: “Stringency,” as used herein is a function of nucleic acid molecule probe length, nucleic acid molecule probe composition (G+C content), salt concentration, organic solvent concentration and temperature of hybridization and/or wash conditions. Stringency is typically measured by the parameter Tₘ, which is the temperature at which 50% of the complementary nucleic acid molecules in the hybridization assay are hybridized, in terms of a temperature differential from Tₘ. High stringency conditions are those providing a condition of Tₘ-5° C. to Tₘ-10° C. Medium or moderate stringency conditions are those providing Tₘ-20° C. to Tₘ-29° C. Low stringency conditions are those providing a condition of Tₘ-40° C. to Tₘ-48° C. The relationship between hybridization conditions and Tₘ (in ° C.) is expressed in the mathematical equation:
Tₘ=81.5−16.6(log₁₀[Na⁺])+0.41(%G+C)−(600/N) (I)
where N is the number of nucleotides of the nucleic acid molecule probe. This equation works well for probes 14 to 70 nucleotides in length that are identical to the target sequence. The equation below, for Tₘ of DNA-DNA hybrids, is useful for probes having lengths in the range of 50 to greater than 500 nucleotides, and for conditions that include an organic solvent (formamide):
Tₘ=81.5+16.6 log{[Na⁺]/(1+0.7[Na⁺])}+0.41(%G+C)−500/L0.63(%formamide) (II)
where L represents the number of nucleotides in the probe in the hybrid (21). The Tₘ of Equation II is affected by the nature of the hybrid: for DNA-RNA hybrids, Tₘ is 10-15° C. higher than calculated; for RNA-RNA hybrids, Tₘ is 20-25° C. higher. Because the Tₘ decreases about 1° C. for each 1% decrease in homology when a long probe is used (Frischauf et al. (1983) J. Mol Biol, 170: 827-842), stringency conditions can be adjusted to favor detection of identical genes or related family members.

Equation II is derived assuming the reaction is at equilibrium. Therefore, hybridizations according to the present invention are most preferably performed under conditions of probe excess and allowing sufficient time to achieve equilibrium. The time required to reach equilibrium can be shortened by using a hybridization buffer that includes a hybridization accelerator such as dextran sulfate or another high volume polymer.

Stringency can be controlled during the hybridization reaction, or after hybridization has occurred, by altering the salt and temperature conditions of the wash solutions. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.

High stringency hybridizations typically involve hybridization and wash steps. The hybridization step may be performed in aqueous hybridization solution at a temperature between 63° C. and 70° C., more preferably at a temperature between 65° C. and 68° C. and most preferably at a temperature of 65° C. Alternatively, the high stringency hybridization step may be performed in formamide hybridization solution at a temperature between 40° C. and 46° C., at a temperature between 41° C. and 44° C. and most preferably at a temperature of 42° C.

A wash step follows hybridization, and an initial wash is performed with wash solution 1 at 25° C. or 37° C. Following the initial wash, additional washes are performed with wash solution 1 at a temperature between 63° C. and 70° C., more preferably at a temperature between 65° C. and 68° C. and most preferably at a temperature of 65° C. The number of additional wash steps can be 1, 2, 3, 4, 5 or more. The time of both the initial and additional wash steps may be 5 minutes, 10 minutes, 15 minutes, 20 minutes, 25 minutes, 30 minutes, 45 minutes, 1 hour, 1.5 hours, 2 hours or more.

Set forth below are the composition of the hybridization and wash solutions and their components. A person of ordinary skill in the art will recognize that these solutions are typical and exemplary of high stringency hybridization solutions.

| | |
|---|---|
| Aqueous Hybridization | 6X SSC or 6X SSPE |
| Solution: | 0.05% Blotto or 5X Denhardt's Reagent |
| | 100 μg/ml denatured salmon sperm DNA |
| | 0.05% SDS |
| Formamide Hybridization | 50% Formamide |
| Solution: | 6X SSC or 6X SSPE |
| | 0.05% Blotto or 5X Denhardt's Reagent |
| | 100 μg/ml denatured salmon sperm DNA |
| | 0.05% SDS |
| Wash Solution 1: | 2X SSC or SSPE |
| | 0.1% SDS |
| Wash Solution 2: | 0.1X SSC or SSPE |
| | 0.5% SDS |
| 20X SSC: | 175.3 g NaCl |
| | 88.2 g Sodium Citrate |
| | Bring to 800 ml with H₂O |
| | Adjust to pH 7 with 10 n NaOH |
| | Bring to 1L with H₂O |
| 20X SSPE: | 175.3 g NaCl |
| | 27.6 g NaH₂PO₄ |
| | Bring to 800 ml with H₂O•H₂O |
| | 7.4 g EDTA |
| | Adjust to pH 7.4 with 10 n NaOH |
| | Bring to 1L with H₂O |
| 1X BLOTTO: | 5% Non-fat dry milk |
| | 0.02% Sodium azide |
| 50X Denhardts's Reagent: | 5 g Ficoll |
| | 5 g Polyvinylpyrrolidone |
| | 5 g BSA |
| | Adjust to 500 ml with H₂O |

Superpool: As used in the context of the current invention, a “superpool” contains an equal amount of seed from 500 different events, representing 100 distinct exogenous nucleotide sequences. An event is a plant carrying a unique insertion of a distinct exogenous sequence which misexpresses that sequence. Transformation of a single polynucleotide sequence can result in multiple events because the sequence can insert in a different part of the genome with each transformation.

T₀: The term “T₀” refers to the whole plant, explant or callus tissue, inoculated with the transformation medium.

T₁: The term T₁, refers to either the progeny of the T₀ plant, in the case of whole-plant transformation, or the regenerated seedling in the case of explant or callous tissue transformation.

T₂: The term T₂ refers to the progeny of the T₁, plant. T₂ progeny are the result of self-fertilization or cross-pollination of a T₁, plant.

T₃: The term T₃ refers to second generation progeny of the plant that is the direct result of a transformation experiment. T₃ progeny are the result of self-fertilization or cross-pollination of a T₂ plant.

Transformation: Examples of means by which this can be accomplished are described below and include Agrobacterium-mediated transformation (of dicots (Needleman and Wunsch (1970) J. Mol. Biol. 48:443; Pearson and Lipman (1988) Proc. Natl. Acad. Sci. (USA) 85: 2444), of monocots (Yamauchi et al. (1996) Plant Mol Biol. 30:321-9; Xu et al. (1995) Plant Mol. Biol 27:237; Yamamoto et al. (1991) Plant Cell 3:371), and biolistic methods (P. Tijessen, “Hybridization with Nucleic Acid Probes” In Laboratory Techniques in Biochemistry and Molecular Biology, P. C. vand der Vliet, ed., c. 1993 by Elsevier, Amsterdam), electroporation, in planta techniques, and the like. Such a plant containing the exogenous nucleic acid is referred to here as a T₀ for the primary transgenic plant and T₁, for the first generation.

Varying Nitrogen Conditions: In the context of the instant invention, the phrase “varying nitrogen conditions” refers to growth conditions where the concentration of available nitrogen fluctuates within and outside of the normal range. This phrase encompasses situations where the available nitrogen concentration is initially low, but increases to normal or high levels as well as situations where the initial available nitrogen concentration is high, but then falls to normal or low levels. Situations involving multiple changes in available nitrogen concentration, such as fluctuations from low to high to low levels, are also encompassed by this phrase. These available nitrogen concentration changes can occur in a gradual or punctuated manner.

3. Important Characteristic of the Polynucleotides and Polypeptides of the Invention

The nucleic acid molecules and polypeptides of the present invention are of interest because when the nucleic acid molecules are mis-expressed (i.e., when expressed at a non-natural location or in an increased or decreased amount relative to wild-type) they produce plants that exhibit improved NUE as compared to wild-type plants grown under normal and/or abnormal nitrogen conditions, as evidenced by the results of various experiments disclosed below. This trait can be used to exploit or maximize plant products. For example, the nucleic acid molecules and polypeptides of the present invention are used to increase the expression of genes that cause the plant to have modulated NUE, biomass, growth rate or seedling vigor.

Because the disclosed sequences and methods increase NUE, vegetative growth and growth rate under normal and/or abnormal nitrogen conditions, the disclosed methods can be used to enhance biomass production. For example, plants that grow vegetatively have an increase in NUE, resulting in improved biomass production when grown under normal and/or abnormal nitrogen conditions, compared to a plant of the same species that is not genetically modified grown under identical conditions. Examples of increases in biomass production include increases of at least 5%, at least 20%, or even at least 50%, when compared to an amount of biomass production by a plant of the same speciesgrown under identical normal and/or abnormal nitrogen conditions.

Preferably, transformed plants are evaluated for the desired low nitrogen tolerance phenotype by comparing the seedling areas or photosynthetic efficiency of transformed and control plants grown for approximately fourteen days. Transformed events with statistically significant differences from controls can be selected or screened.

The life cycle of flowering plants in general can be divided into three growth phases: vegetative, inflorescence, and floral (late inflorescence phase). In the vegetative phase, the shoot apical meristem (SAM) generates leaves that later will ensure the resources necessary to produce fertile offspring. Upon receiving the appropriate environmental and developmental signals the plant switches to reproductive growth and the SAM enters the inflorescence phase (I) and gives rise to an inflorescence with flower primordia. During this phase the fate of the SAM and the secondary shoots that arise in the axils of the leaves is determined by a set of meristem identity genes, some of which prevent and some of which promote the development of floral meristems. Once established, the plant enters the late inflorescence phase where the floral organs are produced. If the appropriate environmental and developmental signals the plant needs to switch to floral, or reproductive, growth are disrupted, the plant will not be able to enter reproductive growth, therefore maintaining vegetative growth.

Seedling vigor is an important characteristic that can greatly influence successful growth of a plant, such as crop plants. Adverse environmental conditions, such as poor or excessive nitrogen availability, dry, wet, cold or hot conditions, can affect a plant's growth cycle, and the vigor of seedlings (i.e. vitality and strength under such conditions can differentiate between successful and failed crop growth). Seedling vigor has often been defined to comprise the seed properties that determine “the potential for rapid, uniform emergence and development of normal seedlings under a wide range of field conditions”. Hence, it would be advantageous to develop plant seeds with increased vigor.

For example, increased seedling vigor would be advantageous for cereal plants such as rice, maize, wheat, etc. production. For these crops, growth can often be slowed or stopped by cool environmental temperatures or limited nitrogen availability during the planting season. In addition, rapid emergence and tillering of rice would permit growers to initiate earlier flood irrigation which can save water and suppress weak growth. Genes associated with increased seed vigor and/or cold tolerance and/or nitrogen tolerance have been sought for producing improved crop varieties.(Walia et al (2005) Plant Physiology 139:822-835)

The nitrogen responsive nucleic acids of the invention also down-regulate genes that lead to feedback inhibition of nitrogen uptake and reduction. Examples of such genes are those encoding the 14-3-3 proteins, which repress nitrate reductase (Swiedrych et al. (2002) J Agric Food Chem 50 (7):2137-41,).

Antisense expression of these in transgenic plants causes an increase in amino acid content and protein content in the seed and/or leaves. Such plants are especially useful for livestock feed. For example, an increase in amino acid and/or protein content in alfalfa provides an increase in forage quality and thus enhanced nutrition.

4. The Polypeptides/Polynucleotides of the Invention

The polynucleotides of the present invention and the proteins expressed via translation of these polynucleotides are set forth in the Sequence Listing, specifically SEQ ID NOS: 80-153 and 155-204. The Sequence Listing also consists of functionally comparable proteins. Polypeptides comprised of a sequence within and defined by one of the consensus sequences can be utilized for the purposes of the invention, namely to make transgenic plants with improved NUE, modulated and improved biomass, growth rate and/or seedling vigor when grown under normal and/or abnormal nitrogen conditions.

5. Use of the Polypeptides to Make Transgenic Plants

To use the sequences of the present invention or a combination of them or parts and/or mutants and/or fusions and/or variants of them, recombinant DNA constructs are prepared that comprise the polynucleotide sequences of the invention inserted into a vector and that are suitable for transformation of plant cells. The construct can be made using standard recombinant DNA techniques (see, Sambrook et al., Molecular Cloning: A Laboratory Manual, Second Edition, Cold Spring Harbor Laboratory Press, 1989, New York.) and can be introduced into the plant species of interest by, for example, Agrobacterium-mediated transformation, or by other means of transformation, for example, as disclosed below.

The vector backbone may be any of those typically used in the field such as plasmids, viruses, artificial chromosomes, BACs, YACs, PACs and vectors such as, for instance, bacteria-yeast shuttle vectors, lambda phage vectors, T-DNA fusion vectors and plasmid vectors (see, Shizuya et al. (1992) Proc. Natl. Acad. Sci. USA, 89: 8794-8797; Hamilton et al. (1996) Proc. Natl. Acad. Sci. USA, 93: 9975-9979; Burke et al. (1987) Science, 236:806-812; Sternberg N. et al. (1990) Proc Natl Acad Sci U S A., 87:103-7; Bradshaw et al. (1995) Nucl Acids Res, 23: 4850-4856; Frischauf et al. (1983) J. Mol Biol, 170: 827-842; Huynh et al., Glover NM (ed) DNA Cloning: A practical Approach, Vol.1 Oxford: IRL Press (1985); Walden et al. (1990) Mol Cell Biol 1: 175-194).

Typically, the construct comprises a vector containing a nucleic acid molecule of the present invention with any desired transcriptional and/or translational regulatory sequences such as, for example, promoters, UTRs, and 3′ end termination sequences. Vectors may also include, for example, origins of replication, scaffold attachment regions (SARs), markers, homologous sequences, and introns. The vector may also comprise a marker gene that confers a selectable phenotype on plant cells. The marker may preferably encode a biocide resistance trait, particularly antibiotic resistance, such as resistance to, for example, kanamycin, bleomycin, or hygromycin, or herbicide resistance, such as resistance to, for example, glyphosate, chlorosulfuron or phosphinotricin.

It will be understood that more than one regulatory region may be present in a recombinant polynucleotide, e.g., introns, enhancers, upstream activation regions, transcription terminators, and inducible elements. Thus, more than one regulatory region can be operably linked to said sequence.

To “operably link” a promoter sequence to a sequence, the translation initiation site of the translational reading frame of said sequence is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is a cis-regulatory element (−212 to −154) from the upstream region of the octopine synthase (ocs) gene (Fromm et al. (1989) The Plant Cell 1:977-984).

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

The choice of promoters to be included depends upon several factors, including, but not limited to, efficiency, selectability, inducibility, desired expression level, and cell- or tissue-preferential expression. It is a routine matter for one of skill in the art to modulate the expression of a sequence by appropriately selecting and positioning promoters and other regulatory regions relative to said sequence.

Some suitable promoters initiate transcription only, or predominantly, in certain cell types. For example, a promoter that is active predominantly in a reproductive tissue (eg., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano et al. (1989) Plant Cell 1:855-866; Bustos et al. (1989) Plant Cell 1:839-854; Green et al. (1988) EMBO J. 7:4035-4044; Meier et al. (1991)Plant Cell 3:309-316; and Zhang et al. (1996) Plant Physiology 110: 1069-1079.

Examples of various classes of promoters are described below. Some of the promoters indicated below are described in more detail in U.S. patent application Ser. Nos. 60/505,689; 60/518,075; 60/544,771; 60/558,869; 60/583,691; 60/619,181; 60/637,140; 10/950,321; 10/957,569; 11/058,689; 11/172,703; 11/208,308; and PCT/US05/23639. It will be appreciated that a promoter may meet criteria for one classification based on its activity in one plant species, and yet meet criteria for a different classification based on its activity in another plant species.

Other Regulatory Regions: A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, eg., a nopaline synthase termination sequence.

Various promoters can be used to drive expression of the polynucleotides of the present invention. Nucleotide sequences of such promoters are set forth in SEQ ID NOS: 1-79. Some of them can be broadly expressing promoters, others may be more tissue preferential.

A promoter can be said to be “broadly expressing” when it promotes transcription in many, but not necessarily all, plant tissues or plant cells. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO: 76), YP0144 (SEQ ID NO: 55), YP0190 (SEQ ID NO: 59), p13879 (SEQ ID NO: 75), YP0050 (SEQ ID NO: 35), p32449 (SEQ ID NO: 77), 21876 (SEQ ID NO: 1), YP0158 (SEQ ID NO: 57), YP0214 (SEQ ID NO: 61), YP0380 (SEQ ID NO: 70), PT0848 (SEQ ID NO: 26), and PT0633 (SEQ ID NO: 7). Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Root-active promoters drive transcription in root tissue, e.g., root endodermis, root epidermis, or root vascular tissues. In some embodiments, root-active promoters are root-preferential promoters, i.e., drive transcription only or predominantly in root tissue. Root-preferential promoters include the YP0128 (SEQ ID NO: 52), YP0275 (SEQ ID NO: 63), PT0625 (SEQ ID NO: 6), PT0660 (SEQ ID NO: 9), PT0683 (SEQ ID NO: 14), and PT0758 (SEQ ID NO: 22). Other root-preferential promoters include the PT0613 (SEQ ID NO: 5), PT0672 (SEQ ID NO: 11), PT0688 (SEQ ID NO: 15), and PT0837 (SEQ ID NO: 24), which drive transcription primarily in root tissue and to a lesser extent in ovules and/or seeds. Other examples of root-preferential promoters include the root-specific subdomains of the CaMV 35S promoter (Lam et al. (1989) Proc. Natl. Acad. Sci. USA 86:7890-7894), root cell specific promoters reported by Conkling et al. (1990) Plant Physiol. 93:1203-1211, and the tobacco RD2 gene promoter.

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin gene promoter (Bustos et al. (1989) Plant Cell 1(9):839-853), the soybean trypsin inhibitor promoter (Riggs et al. (1989) Plant Cell 1(6):609-621), the ACP promoter (Baerson et al. (1993) Plant Mol Biol, 22(2):255-267), the stearoyl-ACP desaturase gene (Slocombe et al. (1994) Plant Physiol 104(4):167-176), the soybean α′ subunit of β-conglycinin promoter (Chen et al. (1986) Proc Natl Acad Sci USA 83:8560-8564), the oleosin promoter (Hong et al. (1997) Plant Mol Biol 34(3):549-555), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al. (1993) Mol. Cell Biol. 13:5829-5842), the beta-amylase gene promoter, and the barley hordein gene promoter. Other maturing endosperm promoters include the YP0092 (SEQ ID NO: 38), PT0676 (SEQ ID NO: 12), and PT0708 (SEQ ID NO: 17.

Promoters that drive transcription in ovary tissues such as the ovule wall and mesocarp can also be useful, e.g., a polygalacturonidase promoter, the banana TRX promoter, and the melon actin promoter. Other such promoters that drive gene expression preferentially in ovules are YP0007 (SEQ ID NO: 30), YP0111 (SEQ ID NO: 46), YP0092 (SEQ ID NO: 38), YP0103 (SEQ ID NO: 43), YP0028 (SEQ ID NO: 33), YP0121 (SEQ ID NO: 51), YP0008 (SEQ ID NO: 31), YP0039 (SEQ ID NO: 34), YP0115 (SEQ ID NO: 47), YP0119 (SEQ ID NO: 49), YP0120 (SEQ ID NO: 50) and YP0374 (SEQ ID NO: 68).

In some other embodiments of the present invention, embryo sac/early endosperm promoters can be used in order drive transcription of the sequence of interest in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF129516); Arabidopsis MEA; Arabidopsis FIS2 (GenBank No. AF096096); and FIE 1.1 (U.S. Pat. No. 6,906,244). Other promoters that may be suitable include those derived from the following genes: maize MACI (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039 (SEQ ID NO: 34), YP0101 (SEQ ID NO: 41), YP0102 (SEQ ID NO: 42), YP0110 (SEQ ID NO: 45), YP0117 (SEQ ID NO: 48), YP0119 (SEQ ID NO: 49), YP0137 (SEQ ID NO: 53), DME, YP0285 (SEQ ID NO: 64), and YP0212 (SEQ ID NO: 60). Other promoters that may be useful include the following rice promoters: p530c10, pOsFIE2-2, pOsMEA, pOsYp102, and pOsYp285.

Promoters that preferentially drive transcription in zygotic cells following fertilization can provide embryo-preferential expression and may be useful for the present invention. Most suitable are promoters that preferentially drive transcription in early stage embryos prior to the heart stage, but expression in late stage and maturing embryos is also suitable. Embryo-preferential promoters include the barley lipid transfer protein (Ltpl) promoter (Plant Cell Rep (2001) 20:647-654, YP0097 (SEQ ID NO: 40), YP0107 (SEQ ID NO: 44), YP0088 (SEQ ID NO: 37), YP0143 (SEQ ID NO: 54), YP0156 (SEQ ID NO: 56), PT0650 (SEQ ID NO: 8), PT0695 (SEQ ID NO: 16), PT0723 (SEQ ID NO: 19), PT0838 (SEQ ID NO: 25), PT0879 (SEQ ID NO: 28) and PT0740 (SEQ ID NO: 20).

Promoters active in photosynthetic tissue in order to drive transcription in green tissues such as leaves and stems are of particular interest for the present invention. Most suitable are promoters that drive expression only or predominantly such tissues. Examples of such promoters include the ribulose-1,5-bisphosphate carboxylase (RbcS) promoters such as the RbcS promoter from eastern larch (Larix laricina), the pine cab6 promoter (Yamamoto et al. (1994) Plant Cell Physiol. 35:773-778), the Cab-1 gene promoter from wheat (Fejes et al. (1990) Plant Mol. Biol. 15:921-932), the CAB-1 promoter from spinach (Lubberstedt et al. (1994) Plant Physiol. 104:997-1006), the cab1R promoter from rice (Luan et al. (1992) Plant Cell 4:971-981), the pyruvate orthophosphate dikinase (PPDK) promoter from corn (Matsuoka et al. (1993) Proc Natl Acad. Sci USA 90:9586-9590), the tobacco Lhcb1*2 promoter (Cerdan et al. (1997) Plant Mol. Biol. 33:245-255), the Arabidopsis thaliana SUC2 sucrose-H+symporter promoter (Truernit et al. (1995) Planta 196:564-570), and thylakoid membrane protein promoters from spinach (psaD, psaF, psaE, PC, FNR, atpC, atpD, cab, rbcS. Other promoters that drive transcription in stems, leafs and green tissue are PT0535 (SEQ ID NO: 3), PT0668 (SEQ ID NO: 2), PT0886 (SEQ ID NO: 29), PR0924 (SEQ ID NO: 78), YP0144 (SEQ ID NO: 55), YP0380 (SEQ ID NO: 70) and PT0585 (SEQ ID NO: 4).

In some other embodiments of the present invention, inducible promoters may be desired. Inducible promoters drive transcription in response to external stimuli such as chemical agents or environmental stimuli. For example, inducible promoters can confer transcription in response to hormones such as giberellic acid or ethylene, or in response to light or drought. Examples of drought inducible promoters are YP0380 (SEQ ID NO: 70), PT0848 (SEQ ID NO: 26), YP0381 (SEQ ID NO: 71), YP0337 (SEQ ID NO: 66), YP0337 (SEQ ID NO: 66), PT0633 (SEQ ID NO: 7), YP0374 (SEQ ID NO: 68), PT0710 (SEQ ID NO: 18), YP0356 (SEQ ID NO: 67), YP0385 (SEQ ID NO: 73), YP0396 (SEQ ID NO: 74), YP0384 (SEQ ID NO: 72), YP0384 (SEQ ID NO: 72), PT0688 (SEQ ID NO: 15), YP0286 (SEQ ID NO: 65), YP0377 (SEQ ID NO: 69), and PD1367 (SEQ ID NO: 79). Examples of promoters induced by nitrogen are PT0863 (SEQ ID NO: 27), PT0829 (SEQ ID NO: 23), PT0665 (SEQ ID NO: 10) and PT0886 (SEQ ID NO: 29). An example of a shade inducible promoter is PR0924 (SEQ ID NO: 78) and an example of a promoter induced by nitrogen deficiency is PT0959 (SEQ ID NO: 154).

Other Promoters: Other classes of promoters include, but are not limited to, leaf-preferential, stem/shoot-preferential, callus-preferential, guard cell-preferential, such as PT0678 (SEQ ID NO: 13), and senescence-preferential promoters. Promoters designated YP0086 (SEQ ID NO: 36), YP0188 (SEQ ID NO: 58), YP0263 (SEQ ID NO: 62), PT0758 (SEQ ID NO: 22), PT0743 (SEQ ID NO: 21), PT0829 (SEQ ID NO: 23), YP0119 (SEQ ID NO: 49), and YP0096 (SEQ ID NO: 39), as described in the above-referenced patent applications, may also be useful.

Alternatively, misexpression can be accomplished using a two component system, whereby the first component consists of a transgenic plant comprising a transcriptional activator operatively linked to a promoter and the second component consists of a transgenic plant that comprise a nucleic acid molecule of the invention operatively linked to the target-binding sequence/region of the transcriptional activator. The two transgenic plants are crossed and the nucleic acid molecule of the invention is expressed in the progeny of the plant. In another alternative embodiment of the present invention, the misexpression can be accomplished by having the sequences of the two component system transformed in one transgenic plant line.

Another alternative consists in inhibiting expression of a biomass or vigor-modulating polypeptide in a plant species of interest. The term “expression” refers to the process of converting genetic information encoded in a polynucleotide into RNA through transcription of the polynucleotide (i.e., via the enzymatic action of an RNA polymerase), and into protein, through translation of mRNA. “Up-regulation” or “activation” refers to regulation that increases the production of expression products relative to basal or native states, while “down-regulation” or “repression” refers to regulation that decreases production relative to basal or native states.

A number of nucleic-acid based methods, including anti-sense RNA, ribozyme directed RNA cleavage, and interfering RNA (RNAi) can be used to inhibit protein expression in plants. Antisense technology is one well-known method. In this method, a nucleic acid segment from the endogenous gene is cloned and operably linked to a promoter so that the antisense strand of RNA is transcribed. The recombinant vector is then transformed into plants, as described above, and the antisense strand of RNA is produced. The nucleic acid segment need not be the entire sequence of the endogenous gene to be repressed, but typically will be substantially identical to at least a portion of the endogenous gene to be repressed. Generally, higher homology can be used to compensate for the use of a shorter sequence. Typically, a sequence of at least 30 nucleotides is used (eg., at least 40, 50, 80, 100, 200, 500 nucleotides or more).

Thus, for example, an isolated nucleic acid provided herein can be an antisense nucleic acid to one of the aforementioned nucleic acids encoding a biomass-modulating polypeptide. A nucleic acid that decreases the level of a transcription or translation product of a gene encoding a biomass-modulating polypeptide is transcribed into an antisense nucleic acid similar or identical to the sense coding sequence of the biomass- or growth rate-modulating polypeptide. Alternatively, the transcription product of an isolated nucleic acid can be similar or identical to the sense coding sequence of a biomass growth rate-modulating polypeptide, but is an RNA that is unpolyadenylated, lacks a 5′ cap structure, or contains an unsplicable intron.

In another method, a nucleic acid can be transcribed into a ribozyme, or catalytic RNA, that affects expression of an mRNA. (See, U.S. Pat. No. 6,423,885). Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al. (1995) Proc. Natl. Acad. Sci. USA, 92(13):6175-6179; de Feyter and Gaudron, Methods in Molecular Biology, Vol. 74, Chapter 43, “Expressing Ribozymes in Plants”, Edited by Turner, P.C, Humana Press Inc., Totowa, N.J. RNA endoribonucleases such as the one that occurs naturally in Tetrahymena thermophila, and which have been described extensively by Cech and collaborators can be useful. See, for example, U.S. Pat. No. 4,987,071.

Methods based on RNA interference (RNAi) can be used. RNA interference is a cellular mechanism to regulate the expression of genes and the replication of viruses. This mechanism is thought to be mediated by double-stranded small interfering RNA molecules. A cell responds to such a double-stranded RNA by destroying endogenous mRNA having the same sequence as the double-stranded RNA. Methods for designing and preparing interfering RNAs are known to those of skill in the art; see, e.g., WO 99/32619 and WO 01/75164. For example, a construct can be prepared that includes a sequence that is transcribed into an interfering RNA. Such an RNA can be one that can anneal to itself, eg., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises an antisense sequence of the biomass-modulating polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. See, e.g., WO 99/53050.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev., 7:187-195; Hyrup et al. (1996) Bioorgan. Med. Chem., 4: 5-23. In addition, the deoxyphosphate backbone can be replaced with, for example, a phosphorothioate or phosphorodithioate backbone, a phosphoroamidite, or an alkyl phosphotriester backbone.

Transformation

Nucleic acid molecules of the present invention may be introduced into the genome or the cell of the appropriate host plant by a variety of techniques. These techniques, able to transform a wide variety of higher plant species, are well known and described in the technical and scientific literature (see, e.g., Weising et al. (1988) Ann. Rev. Genet, 22:421 and Christou (1995) Euphytica, 85:13-27).

A variety of techniques known in the art are available for the introduction of DNA into a plant host cell. These techniques include transformation of plant cells by injection (Newell (2000)), microinjection (Griesbach (1987) Plant Sci. 50:69-77), electroporation of DNA (Fromm et al. (1985) Proc. Natl. Acad. Sci USA 82:5824), PEG (Paszkowski et al. (1984) EMBO J. 3:2717), use of biolistics (Klein et al. (1987) Nature 327:773), fusion of cells or protoplasts (Willmitzer, L. (1993) Transgenic Plants. In: lotechnology, A Multi-Volume Comprehensive treatise (H.J. Rehm, G. Reed, A. Püler, P. Stadler, eds., Vol. 2, 627-659, VCH Weinheim-New York-Basel-Cambridge), and via T-DNA using Agrobacterium tumefaciens (Crit. Rev. Plant. Sci. 4:1-46; Fromm et al. (1990) Biotechnology 8:833-844) or Agrobacterium rhizogenes (Cho et al. (2000) Planta 210:195-204) or other bacterial hosts (Brootghaerts et al. (2005) Nature 433:629-633), for example.

In addition, a number of non-stable transformation methods that are well known to those skilled in the art may be desirable for the present invention. Such methods include, but are not limited to, transient expression (Lincoln et al. (1998) Plant Mol. Biol. Rep. 16:1-4) and viral transfection (Lacomme et al. (2001), “Genetically Engineered Viruses” (C.J.A. Ring and E.D. Blair, Eds). Pp. 59-99, BIOS Scientific Publishers, Ltd. Oxford, UK).

Seeds are obtained from the transformed plants and used for testing stability and inheritance. Generally, two or more generations are cultivated to ensure that the phenotypic feature is stably maintained and transmitted.

A person of ordinary skill in the art recognizes that after the expression cassette is stably incorporated in transgenic plants and confirmed to be operable, it can be introduced into other plants by sexual crossing. Any of a number of standard breeding techniques can be used, depending upon the species to be crossed.

The nucleic acid molecules of the present invention may be used to confer the trait of improved NUE, including improved tolerance to high or low nitrogen conditions. The invention has utility in improving important agronomic characteristics of crop plants, for example enabling plants to be productively cultivated with lower nitrogen fertilizer inputs and on nitrogen-poor soil. As noted above, transformed plants that exhibit overexpression of the polynucleotides of the invention grow well under low nitrogen conditions and exhibit increased tolerance to varying nitrogen conditions. These require less fertilizer, leading to lower costs for the farmer and reduced nitrate pollution of ground water.

In aspects related to making transgenic plants, a typical step involves selection or screening of transformed plants, e.g., for the presence of a functional vector as evidenced by expression of a selectable marker. Selection or screening can be carried out among a population of recipient cells to identify transformants using selectable marker genes such as herbicide resistance genes. Physical and biochemical methods can be used to identify transformants. These include Southern analysis or PCR amplification for detection of a polynucleotide; Northern blots, SI RNase protection, primer-extension, or RT-PCR amplification for detecting RNA transcripts; enzymatic assays for detecting enzyme or ribozyme activity of polypeptides and polynucleotides; and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

A population of transgenic plants can be screened and/or selected for those members of the population that have a desired trait or phenotype conferred by expression of the transgene. For example, a population of progeny of a single transformation event can be screened for those plants having a desired level of expression of a heterologous NUE-modulating polypeptide or nucleic acid. As an alternative, a population of plants comprising independent transformation events can be screened for those plants having a desired trait, such as NUE. Selection and/or screening can be carried out over one or more generations, which can be useful to identify those plants that have a statistically significant difference in a protein level as compared to a corresponding level in a control plant. Selection and/or screening can also be carried out in more than one geographic location. In some cases, transgenic plants can be grown and selected under conditions which induce a desired phenotype or are otherwise necessary to produce a desired phenotype in a transgenic plant. In addition, selection and/or screening can be carried out during a particular developmental stage in which the phenotype is expected to be exhibited by the plant. Selection and/or screening can be carried out to choose those transgenic plants having a statistically significant difference in NUE relative to a control plant that lacks the transgene. Selected or screened transgenic plants have an altered phenotype as compared to a corresponding control plant, as described in the “Important Characteristics of the Polynucleotides of the Invention” section above.

Generally, the polynucleotides and polypeptides of the invention can be used to improve plant performance when plants are grown under sub-optimal, normal or abnormal nitrogen conditions. For example, the transgenic plants of the invention can be grown without damage on soils or solutions containing at least 1, 2, 3, 4 or 5 percent less nitrogen, more preferably at least 5, 10, 20, 30, 40 or 50 percent less nitrogen, even more preferably at least 60, 70 or 80 percent less nitrogen and most preferably at least 90 or 95 percent less nitrogen than normally required for a particular plant species/crop, depending on the promoter or promoter control element used. Similarly, the transgenic plants of the invention can be grown without damage on soils or solutions containing at least 1, 2, 3, 4 or 5 percent more nitrogen, more preferably at least 5, 10, 20, 30, 40 or 50 percent more nitrogen, even more preferably at least 60, 70 or 80 percent more nitrogen and most preferably at least 90 or 95 percent more nitrogen than normally tolerated for a particular plant species/crop, depending on the promoter or promoter control element used.

The nucleic acid molecules of the present invention encode appropriate proteins from any organism, but are preferably found in plants, fungi, bacteria or animals.

Transgenic Plant Phenotypes

Information that the polypeptides disclosed herein can modulate nitrogen use efficiency is useful in breeding of crop plants. Based on the effect of the disclosed polypeptides on nitrogen use efficiency, one can search for and identify polymorphisms linked to genetic loci for such polypeptides. Polymorphisms that can be identified include simple sequence repeats (SSRs), amplified fragment length polymorphisms (AFLPs) and restriction fragment length polymorphisms (RFLPs).

If a polymorphism is identified, its presence and frequency in populations is analyzed to determine if it is statistically significantly correlated to an increase in nitrogen use efficiency. Those polymorphisms that are correlated with an increase in nitrogen use efficiency can be incorporated into a marker assisted breeding program to facilitate the development of lines that have a desired increase in nitrogen use efficiency. Typically, a polymorphism identified in such a manner is used with polymorphisms at other loci that are also correlated with a desired increase in nitrogen use efficiency or other desired trait.

The methods according to the present invention can be applied to any plant, preferably higher plants, pertaining to the classes of Angiospermae and Gymnospermae. Plants of the subclasses of the Dicotylodenae and the Monocotyledonae are particularly suitable. Dicotyledonous plants belonging to the orders of the Magniolales, Illiciales, Laurales, Piperales Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, Santales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales, for example, are also suitable. Monocotyledonous plants belonging to the orders of the Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchidales also may be useful in embodiments of the present invention. Further examples include, but are not limited to, plants belonging to the class of the Gymnospermae are Pinales, Ginkgoales, Cycadales and Gnetales.

The methods of the present invention are preferably used in plants that are important or interesting for agriculture, horticulture, biomass for bioconversion and/or forestry. Non-limiting examples include, for instance, tobacco, oilseed rape, sugar beet, potatoes, tomatoes, cucumbers, peppers, beans, peas, citrus fruits, avocados, peaches, apples, pears, berries, plumbs, melons, eggplants, cotton, soybean, sunflowers, roses, poinsettia, petunia, guayule, cabbages, spinach, alfalfa, artichokes, sugarcane, mimosa, Servicea lespedera, corn, wheat, rice, rye, barley, sorghum and grasses such as switch grass, giant reed, Bermuda grass, Johnson grasses or turf grass, millet, hemp, bananas, poplars, eucalyptus trees and conifers. Of interest are plants grown for energy production, so called energy crops, such as broadleaf plants like alfalfa, hemp, Jerusalem artichoke and grasses such as sorghum, switchgrass, Johnson grass and the likes. Thus, the described materials and methods are useful for modifying biomass characteristics, such as characteristics of biomass renewable energy source plants. A biomass renewable energy source plant is a plant having or producing material (either raw or processed) that comprises stored solar energy that can be converted to fuel. In general terms, such plants comprise dedicated energy crops as well as agricultural and woody plants. Examples of biomass renewable energy source plants include: switchgrass, elephant grass, giant chinese silver grass, energycane, giant reed (also known as wild cane), tall fescue, bermuda grass, sorghum, napier grass, also known as uganda grass, triticale, rye, winter wheat, shrub poplar, shrub willow, big bluestem, reed canary grass and corn.

Homologues Encompassed by the Invention

It is known in the art that one or more amino acids in a sequence can be substituted with other amino acid(s), the charge and polarity of which are similar to that of the substituted amino acid, ie. a conservative amino acid substitution, resulting in a biologically/functionally silent change. Conservative substitutes for an amino acid within the polypeptide sequence can be selected from other members of the class to which the amino acid belongs. Amino acids can be divided into the following four groups: (1) acidic (negatively charged) amino acids, such as aspartic acid and glutamic acid; (2) basic (positively charged) amino acids, such as arginine, histidine, and lysine; (3) neutral polar amino acids, such as serine, threonine, tyrosine, asparagine, and glutamine; and (4) neutral nonpolar (hydrophobic) amino acids such as glycine, alanine, leucine, isoleucine, valine, proline, phenylalanine, tryptophan, cysteine, and methionine.

Nucleic acid molecules of the present invention can comprise sequences that differ from those encoding a protein or fragment thereof selected from the group consisting of Leads 82, 85, 92, 93, 98, 112, ME07344, ME05213, ME02730 and ME24939, corresponding to SEQ ID NOS: 81, 105, 107, 114, 116, 201, 140, 84, 112 and 200, respectively, due to the fact that the different nucleic acid sequence encodes a protein having one or more conservative amino acid changes.

Biologically functional equivalents of the polypeptides, or fragments thereof, of the present invention can have about 10 or fewer conservative amino acid changes, more preferably about 7 or fewer conservative amino acid changes, and most preferably about 5 or fewer conservative amino acid changes. In a preferred embodiment of the present invention, the polypeptide has between about 5 and about 500 conservative changes, more preferably between about 10 and about 300 conservative changes, even more preferably between about 25 and about 150 conservative changes, and most preferably between about 5 and about 25 conservative changes or between 1 and about 5 conservative changes.

Identification of Useful Nucleic Acid Molecules and Their Corresponding Nucleotide Sequences

The nucleic acid molecules, and nucleotide sequences thereof, of the present invention were identified by use of a variety of screens that are predictive of nucleotide sequences that provide plants with improved NUE, vegetative growth, growth rate, and/or biomass when grown under abnormal nitrogen conditions. One or more of the following screens were, therefore, utilized to identify the nucleotide (and amino acid) sequences of the present invention.

The present invention is further exemplified by the following examples. The examples are not intended to in any way limit the scope of the present application and its uses.

6. Experiments Confirming the Usefulness of the Polynucleotides and Polypeptides of the Invention

General Protocols

Agrobacterium-Mediated Transformation of Arabidopsis

Wild-type Arabidopsis thaliana Wassilewskija (WS) plants are transformed with Ti plasmids containing clones in the sense orientation relative to the 35S promoter. A Ti plasmid vector useful for these constructs, CRS 338, contains the Ceresconstructed, plant selectable marker gene phosphinothricin acetyltransferase (PAT), which confers herbicide resistance to transformed plants.

Ten independently transformed events are typically selected and evaluated for their qualitative phenotype in the T₁, generation.

Preparation of Soil Mixture: 24L SunshineMix #5 soil (Sun Gro Horticulture, Ltd., Bellevue, Wash.) is mixed with 16L Therm-O-Rock vermiculite (Therm-O -Rock West, Inc., Chandler, Ariz.) in a cement mixer to make a 60:40 soil mixture. To the soil mixture is added 2 Tbsp Marathon 1% granules (Hummert, Earth City, Mo.), 3 Tbsp OSMOCOTE® 14-14-14 (Hummert, Earth City, Mo.) and 1 Tbsp Peters fertilizer 20-20-20 (J.R. Peters, Inc., Allentown, Pa.), which are first added to 3 gallons of water and then added to the soil and mixed thoroughly. Generally, 4-inch diameter pots are filled with soil mixture. Pots are then covered with 8-inch squares of nylon netting.

Planting: Using a 60 mL syringe, 35 mL of the seed mixture is aspirated. 25 drops are added to each pot. Clear propagation domes are placed on top of the pots that are then placed under 55% shade cloth and subirrigated by adding 1 inch of water.

Plant Maintenance: 3 to 4 days after planting, lids and shade cloth are removed. Plants are watered as needed. After 7-10 days, pots are thinned to 20 plants per pot using forceps. After 2 weeks, all plants are subirrigated with Peters fertilizer at a rate of 1 Tsp per gallon of water. When bolts are about 5-10 cm long, they are clipped between the first node and the base of stem to induce secondary bolts. Dipping infiltration is performed 6 to 7 days after clipping.

Preparation of Agrobacterium: To 150 mL fresh YEB is added 0.1 mL each of carbenicillin, spectinomycin and rifampicin (each at 100 mg/ml stock concentration). Agrobacterium starter blocks are obtained (96-well block with Agrobacterium cultures grown to an OD₆₀₀ of approximately 1.0) and inoculated one culture vessel per construct by transferring 1 mL from appropriate well in the starter block. Cultures are then incubated with shaking at 27° C. Cultures are spun down after attaining an OD₆₀₀ of approximately 1.0 (about 24 hours). 200 mL infiltration media is added to resuspend Agrobacterium pellets. Infiltration media is prepared by adding 2.2 g MS salts, 50 g sucrose, and 5 μl 2 mg/ml benzylaminopurine to 900 ml water.

Dipping Infiltration: The pots are inverted and submerged for 5 minutes so that the aerial portion of the plant is in the Agrobacterium suspension. Plants are allowed to grow normally and seed is collected.

High-Throughput Phenotypic Screening of T₁, Misexpression Mutants

Seed is evenly dispersed into water-saturated soil in pots and placed into a dark 4° C. cooler for two nights to promote uniform germination. Pots are then removed from the cooler and covered with 55% shade cloth for 4-5 days. Cotyledons are fully expanded at this stage. FINALE® (Sanofi Aventis, Paris, France) is sprayed on plants (3 ml FINALE® diluted into 48 oz. water) and repeated every 3-4 days until only transformants remain.

Screening: Screening is routinely performed at four stages: Seedling, Rosette, Flowering, and Senescence.
Seedling—the time after the cotyledons have emerged, but before the 3^{rd} true leaf begins to form.
Rosette—the time from the emergence of the 3^{rd} true leaf through just before the primary bolt begins to elongate.
Flowering—the time from the emergence of the primary bolt to the onset of senescence (with the exception of noting the flowering time itself, most observations should be made at the stage where approximately 50% of the flowers have opened).
Senescence—the time following the onset of senescence (with the exception of “delayed senescence”, most observations should be made after the plant has completely dried). Seeds are then collected.
   Screening Superpools for Tolerance to Low Nitrate Growth Conditions

Superpools are generated and two thousand seeds each from ten superpools are pooled together and assayed using the Low Nitrate Screen on Agar. Low nitrate growth media, pH 5.7, is as follows: 0.5×MS without N (PhytoTech), 0.5% sucrose (Sigma), 300 μM KNO₃ (Sigma), 0.5 g MES hydrate (Sigma), 0.8% Phytagar (EM Science). 45 ml of media per square plate is used.

Arabidopsis thaliana cv WS seed is sterilized in 50% Clorox™ with 0.01% Triton X-100 (v/v) for five minutes, washed four times with sterile distilled deionized water and stored at 4° C. in the dark for 3 days prior to use.

Seed is plated at a density of 100 seeds per plate. Wild-type seed is used as a control. Plates are incubated in a Conviron™ growth chamber at 22° C. with a 16:8 hour light:dark cycle from a combination of incandescent and fluorescent lamps emitting a light intensity of ˜100 μEinsteins. and 70% humidity.

Seedlings are screened daily after 14 days. Candidate seedlings are larger or stay greener longer relative to wild-type controls. DNA is isolated from each candidate plant and sequenced to determine which transgene was present.

Seedling Low Nitrate Assay on Agar

Media and seeds are prepared as described above.

Seeds from five misexpression line events, each containing the same polynucleotide, are sown in two rows, with ten seeds per row. Each plate contains five events, for a total of 100 seeds. Control plates containing wild-type seed are also prepared. Plates are then incubated at 4° C. for at least two days.

After the several day 4° C. cold treatment, plates are incubated in a Conviron™ growth chamber at 22° C. with a 16:8 hour light:dark cycle from a combination of incandescent and fluorescent lamps emitting a light intensity of ˜100 peinsteins and 70% humidity.

After 14 days, plates are scanned daily using a CF Imager (Technologica Ltd.) with a 45 minute dark acclimation. The CF Imager is used to quantify the seedlings' optimum quantum yields (Fv/Fm) as a measure of photosynthetic health (see details below). To quantify the seedlings' sizes, plates are also scanned with a flatbed photo scanner (Epson) one day after nitrogen stress is apparent and wild-type seedling growth is arrested. Image capture is ended after all wild-type plants have completely yellowed. On the final scanning day plates are uncovered and liberally sprayed with Finale® (10 ml in 48 oz. Murashige & Skoog liquid media) and returned to the growth chamber.

Two days after spraying, the plates are placed in a closed box for 45 minutes to acclimate in preparation for fluorescence visualization via CF Imager. Plants resistant to Finale® appear red while sensitive plants appear blue. After image capture, plants are assigned a transgenic (resistant) or non-transgenic (sensitive) status. The non-transgenic plants (i.e. non-transgenic segregants) serve as internal controls.

Seedling photosynthetic efficiency, or electron transport via photosystem II, is estimated by the relationship between Fm, the maximum fluorescence signal, and the variable fluorescence, Fv. Here, a reduction in the optimum quantum yield (Fv/Fm) indicates stress, and so can be used to monitor the performance of transgenic plants compared to nontransgenic plants under nitrogen stress conditions. Since a large amount of nitrogen is invested in maintaining the photosynthetic apparatus, nitrogen deficiencies can lead to dismantling of the reaction centers and to reductions in photosynthetic efficiency. Consequently, from the start of image capture collection until the plants are dead the Fv/Fm ratio is determined for each seedling using the Flurolmager 2 software (Kevin Oxborough and John Bartington).

The rosette area of each plant is also analyzed using WinRHIZO software (Regent Instruments) to analyze the Epson flatbed scanner captured images.

Low Nitrate Validated Assay

Media and seeds are prepared as described above.

For misexpression lines which pass the above low nitrate assay, both T₂ and T₃ generation seed for an event are plated along with wild-type seed, at a final density of 100 seeds per plate. Plates contain 10 seed/row and have four rows of 10 T₂ seed followed by two rows of wild-type seed, followed by four rows of T₃ seed. Plates are then incubated at 4° C. for at least two days.

After the several day 4° C. cold treatment, plates are incubated in a Conviron™ growth chamber at 22° C. with a 16:8 hour light:dark cycle from a combination of incandescent and fluorescent lamps emitting a light intensity of ˜100 μEinsteins and 70% humidity.

After 14 days, plates are scanned daily using a CF Imager (Technologica Ltd.) with a 45 minute dark acclimation. The CF Imager is used to quantify the seedlings' optimum quantum yields (Fv/Fm) as a measure of photosynthetic health. To quantify the seedlings' sizes, plates are also scanned with a flatbed photo scanner (Epson) one day after nitrogen stress is apparent and wild-type seedling growth is arrested. Image capture is ended after all wild-type plants have completely yellowed. On the final scanning day plates are uncovered and liberally sprayed with Finale® (10 ml in 48 oz. Murashige & Skoog liquid media) and returned to the growth chamber.

Two days after spraying, the plates are placed in a closed box for 45 minutes to acclimate in preparation for fluorescence visualization via CF Imager. Plants resistant to Finale® appear red while sensitive plants appear blue. After image capture, plants are assigned a transgenic (resistant) or non-transgenic (sensitive) status. The non-transgenic plants (i.e. non-transgenic segregants) serve as internal controls.

Fv/Fm ratio is determined for each seedling using the Flurolmager 2 software (Kevin Oxborough and John Bartington).

The rosette area of each plant is also analyzed using WinRHIZO software (Regent Instruments) to analyze the Epson flatbed scanner captured images.

Results:

Plants transformed with the genes of interest were screened as described above for modulated growth and phenotype characteristics. The observations include those with respect to the entire plant, as well as parts of the plant, such as the roots and leaves.

Summary

| | |
|---|---|
| Sub-trait Area: | Low Nitrogen Tolerance — Increased plant growth rate, biomass, |
| | seed set, photosynthesis or harvest index under growth limiting |
| | nitrogen condition |
| Coding sequence/ | 1: Vector Construct Sequence Identifier 14300854 |
| Species of Origin | corresponding to Clone 154343 - ME02507 encodes a 266 |
| | amino acid Myb-like protein from Arabidopsis. |
| | 2: Vector Construct Sequence Identifier |
| | 21992407 corresponding to Clone 346992 - ME10738 encodes a |
| | putative 47 amino acid unknown protein from corn. |
| | 3: Vector Construct Sequence Identifier 22796530 |
| | corresponding to Clone 560731 - ME08309 encodes a 128 |
| | amino acid Zinc Finger C3HC4 transcription factor from |
| | soybean. |
| | 4: Vector Construct Sequence Identifier 21993270 |
| | corresponding to genomic locus At4g24700 - ME10822 encodes |
| | a 143 amino acid protein of unknown function from |
| | Arabidopsis. |
| | 5: Vector Construct Sequence Identifier 14300796 |
| | corresponding to Clone 150823 - ME03926 encodes a 516 |
| | amino acid glycosyl hydrolase family 9 protein from |
| | Arabidopsis |
| | 6: Vector Construct Sequence Identifier 14297694 |
| | corresponding to Clone 14432 - ME07523 encodes a 156 amino |
| | acid bZIP transcription factor from Arabidopsis |
| | 7: Vector Construct Sequence Identifier 14300163 |
| | corresponding to Clone 101255 - ME07344 encodes a 359 |
| | amino acid CCCH-type zinc finger transcription factor from |
| | Arabidopsis |

All of the plants discussed in the Examples below have no observable or statistical differences from wildtype plants with respect to germination rate. Under control of the 35S promoter, Example 3 showed only a slight difference in the number of days to flowering and the area of the rosette 7 days post-bolting.

### EXAMPLE 1

Lead Summary: Lead 82-ME02507 (SEQ ID NO: 81)

| Construct | Event/Generation | Plant Stage | Assay | Result |
|---|---|---|---|---|
| 35S::154343 | −11/T₂ segregating | Seedling | Low Nitrate | Significant at p ≦ .05 |
| | plants | | Tolerance | |
| 35S::154343 | −13/T₂ segregating | Seedling | Low Nitrate | Significant at p ≦ .05 |
| | plants | | Tolerance | |
| 35S::154343 | −11/T₃ segregating | Seedling | Low Nitrate | Significant at p ≦ .05 |
| | plants | | Tolerance | |
| 35S::154343 | −13/T₃ segregating | Seedling | Low Nitrate | Significant at p ≦ .05 |
| | plants | | Tolerance | |

Ectopic expression of Clone 154343 under the control of the 35S promoter induces the following phenotypes:

Enhanced photosynthesis after fourteen days on low nitrate-containing media compared to controls.

ME02507 Was Identified from a Superpool Screen for Tolerance to Low Nitrate Conditions.

Superpools 2-11 and 22-31 were screened for seedlings that were larger or greener than controls on low nitrate growth media (300 μM KNO₃ MS). Transgene sequence was obtained for 17 candidate seedlings from Superpools 2-11. Two of the 17 candidate sequences aligned with ME02507 when analyzed using BLAST. Transgene sequence was also obtained for 39 candidate seedlings from Superpools 22-31. Eight of the 39 candidate sequences aligned with ME02507 when analyzed using BLAST.

Two Events of ME02507 Show 3:1 Segregation for Finale® Resistance.

Events −11 and −13 segregated 3:1 (R:S) for Finale® resistance in the T₂ generation (data not shown).

Two Events of ME02507 Showed Significantly Increased Photosynthetic Efficiency under Low Nitrate Growth Conditions in Both Generations.

Seeds representing three events of ME02507 from each of the T₂ and the T₃ generations were sown on low nitrate growth media (300 μM KNO₃ MS). Two events, −11 and −13, showed a significant increase in photosynthetic efficiency in both generations at p=0.05 when measured using a one-tailed t-test and assuming unequal variance (Table 1-1).

TABLE 1-1
T-test comparison of seedling photosynthetic efficiency
between transgenic seedlings and pooled non-transgenic
segregants after 14 days of growth on low nitrate.

| | | | | Pooled Non- | | |
|---|---|---|---|---|---|---|
| | | Transgenic | | Transgenics | | t-test |
| Line | Events | Fv/Fm | n | Fv/Fm | n | p-value |
| ME02507 | ME02507-11 | 0.60 | 16 | 0.52 | 14 | 0.00079 |
| ME02507 | ME02507-11-99 | 0.62 | 30 | 0.52 | 14 | 5.43E−06 |
| ME02507 | ME02507-13 | 0.59 | 28 | 0.53 | 23 | 0.000103 |
| ME02507 | ME02507-13-99 | 0.61 | 29 | 0.53 | 23 | 3.75E−05 |

Qualitative Analysis of the T₁Plants:

There was no observable difference in the physical appearance of twenty-two of the twenty-four plants compared to the controls. Of the two remaining plants, one from Event −02 was large and late flowering and one from Event −13 was darker green.

Qualitative and Quantitative Analysis of the T₂ Plants:

Events −11 and −13 of ME02507 appeared similar to wild-type to slightly darker green in all instances.

The effect of Lead 82 on NUE was tested by growing plants in soil with a low amount of nitrogen and measuring biomass production after the onset of flowering. Plants were grown in a soil mix consisting of 3:2 metromix 200: vermiculite without any supplemental nitrogen under long day light conditions in a Conviron™ Model TCR growth chamber.

At the mid flowering stage of development total leaf area was measured for ME2507 Event −11 , Event −13 and transgenic control (vector without the Lead 82 cDNA) plants and the entire shoot harvested, dried and weighed to determine shoot dry weight. The results indicate that Lead 82 significantly increased rosette area by 45% and 57% for Events

−11 and −13, respectively, compared to the transgenic control plants (significant at p<0.05 level by t-test). The change in rosette area size translated into a 45% increase in biomass production for Event −13 compared to transgenic control plants (significant at p<0.05 level by t-test). While Event −11 also showed an increase in biomass, this increase was not statistically significant. The data indicate that the low nitrate tolerance Lead 82 can significantly increase nitrogen use efficiency and thus increase rosette area production and biomass production. Transcription factors often control the expression of multiple genes in a pathway. For example, basic helix-loop-helix (bHLH) and Myb transcription factors are thought to be involved in controlling the expression of several genes in a pathway, such as carbon flux through the TCA cycle (Yanagisawa et al., 2004). Several Myb genes have been shown to regulate the structural genes of several pathways, such as the anthocyanin pathway (Sainz et al., 1997; Hernandez et al., 2004). In addition, Myb genes have also been implicated in regulation of gene expression by nitrogen (Todd et al., 2004).

Clone 154343 encodes a Myb transcription factor that confers a “stay green” phenotype under low nitrate assay conditions. Plants mis-expressing clone 154343 also show improved photosystem II electron transport under low nitrate growth conditions compared to wild-type controls and transgene-minus siblings. Plants mis-expressing clone 154343 also show improved nitrogen use efficiency when grown on soil as evidenced by increased leaf area and biomass production under limiting nitrogen fertilizer conditions.

### EXAMPLE 2

Lead Summary: Lead 85- ME10738 (SEQ ID NO: 105)

| Construct | Event/Generation | Plant Stage | Assay | Result |
|---|---|---|---|---|
| 35S::346992 | −03/T₂ segregating | Seedling | Low Nitrate | Significant at p ≦ .05 |
| | plants | | Tolerance | |
| 35S::346992 | −05/T₂ segregating | Seedling | Low Nitrate | Significant at p ≦ .05 |
| | plants | | Tolerance | |
| 35S::346992 | −03/T₃ segregating | Seedling | Low Nitrate | Significant at p ≦ .05 |
| | plants | | Tolerance | |
| 35S::346992 | −05/T₃ segregating | Seedling | Low Nitrate | Significant at p ≦ .05 |

Ectopic expression of Clone 346992 under the control of the 35S promoter induces the following phenotypes:

Enhanced photosynthesis after fourteen days on low nitrate-containing media compared to controls.

ME10738 was Identified from a Superpool Screen for Tolerance to Low Nitrate Conditions.

Superpools 72-81 were screened for seedlings that were larger or greener than controls on low nitrate growth media (300 μM KNO₃ MS). Transgene sequence was obtained for 23 candidate seedlings. One of the 23 candidate sequences aligned with ME10738 when analyzed with BLAST.

One Event of ME10738 Show 3:1 Segregation for Finale® Resistance.

Event −03 segregated 3:1 (R:S) for Finale® resistance in the T₂ generation (data not shown). Event −05 segregated 1:1 (18:17; R:S) for Finale® resistance.

Two Events of ME10738 Showed Significantly Increased Photosynthetic Efficiency under Low Nitrate Growth Conditions in Both Generations.

Seeds representing five events of ME10738 from each of the T₂ and the T₃ generations were sown in the Low Nitrate Assay. Two events, −03 and −05, showed a significant increase in photosynthesis in both generations at p=0.05 as measured using a one-tailed t-test and assuming unequal variance (Table 2-1).

TABLE 2-1
t-test comparison of seedling photosynthetic efficiency
between transgenic seedlings and pooled non-transgenic
segregants after 14 days of growth on low nitrate.

| | | | | Pooled Non- | | |
|---|---|---|---|---|---|---|
| | | Transgenic | | Transgenics | | t-test |
| Line | Events | Fv/Fm | n | Fv/Fm | n | p-value |
| ME10738 | ME10738-03 | 0.60 | 26 | 0.56 | 22 | 0.0405 |
| ME10738 | ME10738-03-99 | 0.62 | 27 | 0.56 | 22 | 0.00237 |
| ME10738 | ME10738-05 | 0.59 | 19 | 0.55 | 24 | 0.0263 |
| ME10738 | ME10738-05-99 | 0.59 | 30 | 0.55 | 24 | 0.0187 |

Qualitative Analysis of the T₁ Plants:

There were no observable differences in the physical appearance of all 10 events compared to the controls.

Qualitative and Quantitative Analysis of the T₂ Plants:

Events −03 and −05 of ME10738 appeared similar to wild-type to slightly darker green in all instances.

Corn clone 346992 encodes a short polypeptide with no significant sequence identity to any known proteins. The sequence maps to a methyl-filtration selected maize genomic sequence, indicating it is hypomethylated and a candidate for residing in an expressed region of the maize genome (ZmGSStuc11-12-04.257770.1). Plants mis-expressing clone 346992 also show improved photosystem II electron transport under low nitrate growth conditions compared to wildtype controls and transgene-minus siblings. The short polypeptide may represent a novel peptide that has a role in nutrient signaling. Alternatively, the cDNA may be derived from a non-protein-coding RNA that may have a role in gene regulation through RNA-based mechanisms (Marker et al. (2002) Curr Biol 12:2002-2013; Tang et al. (2005) Mol Microbiol 55:469-481).

### EXAMPLE 3

Lead Summary: Lead 92-ME08309 (SEQ ID NO: 107)

| Construct | Event/Generation | Plant Stage | Assay | Result |
|---|---|---|---|---|
| 35S::560731 | −02/T₂ segregating | Seedling | Low Nitrate | Significant at p ≦ .05 |
| | plants | | Tolerance | |
| 35S::560731 | −05/T₂ segregating | Seedling | Low Nitrate | Significant at p ≦ .05 |
| | plants | | Tolerance | |
| 35S::560731 | −02/T₃ segregating | Seedling | Low Nitrate | Significant at p ≦ .05 |
| | plants | | Tolerance | |
| 35S::560731 | −05/T₃ segregating | Seedling | Low Nitrate | Significant at p ≦ .05 |
| | plants | | Tolerance | |

Ectopic expression of Clone 560731 under the control of the 35S promoter induces the following phenotypes:

Enhanced photosynthesis after 14 days on low nitrate-containing media compared to controls.

Rosette and cauline leaves remain green longer than controls under standard growth conditions.

ME08309 Was Identified from a Superpool Screen for Tolerance to Low Nitrate Conditions.

Superpools 62-71 were screened for seedlings that were larger or greener than controls on low nitrate growth media (300 μM KNO₃ MS). For Superpools 62-71, transgene sequence was obtained for 20 candidate seedlings. One of the 20 candidate sequences aligned with ME08309 when analyzed with BLAST.

Two Events of ME08309 Show 3:1 Segregation for Finale® Resistance.

Events −02 and −05 segregated 3:1 (R:S) for Finale® resistance in the T₂ generation (data not shown).

Two Events of ME08309 Showed Significantly Increased Photosynthetic Efficiency under Low Nitrate Growth Conditions in Both Generations.

Seeds representing two events of ME08309 from each of the T₂ and the T₃ generations were sown on a low nitrate media (300 μM KNO₃ MS). Two events, −02 and −05, showed a significant increase in photosynthetic efficiency in both generations at p=0.05 as measured using a one-tailed t-test and assuming unequal variance (Table 3-1).

TABLE 3-1
T-test comparison of seedling photosynthetic efficiency
between transgenic seedlings and pooled non-transgenic
segregants after 14 days of growth on low nitrate.

| | | | | Pooled Non- | | |
|---|---|---|---|---|---|---|
| | | Transgenic | | Transgenics | | t-test |
| Line | Events | Fv/Fm | n | Fv/Fm | n | p-value |
| ME08309 | ME08309-02 | 0.63 | 30 | 0.59 | 16 | 0.0115 |
| ME08309 | ME08309-02-99 | 0.65 | 34 | 0.59 | 16 | 0.0007 |
| ME08309 | ME08309-05 | 0.67 | 29 | 0.63 | 8 | 0.0262 |
| ME08309 | ME08309-05-99 | 0.70 | 34 | 0.63 | 8 | 0.0026 |

ME08309 Leaves Stay Green Longer than the Control

Both events, −02 and −05, of ME08309 had rosette and cauline leaves that remained green (i.e. a “stay green” phenotype) when compared to the controls. These plants may be accumulating cytokinins which would contribute to the “stay green” phenotype and would also likely give rise to increased photosynthesis on low nitrate medium. Alternatively, they may be accumulating significantly more nitrate during normal growth which cannot be completely remobilized and so the leaves stay green well into senescence.

Qualitative Analysis of the T₁, Plants:

There were no observable differences in the physical appearance of the ten T₁, plants compared to the controls.

Qualitative and Quantitative Analysis of the T₂ Plants:

There were no observable or statistical differences between events −02 and −05 of ME08309 and wild type plants for germination or fertility (as measured by silique number and seed fill).

General morphology/architecture: Rosette and cauline leaves appear to stay greener for a longer time than the controls.

Days to flowering: Plants may be slightly later flowering than controls.

Rosette area 7 days post-bolting: Rosettes may be slightly smaller than controls.

Clone 560731 encodes a 128 amino acid ring finger protein from the Zinc Finger C3HC4 protein family. The ring finger is a specialized zinc finger protein domain that binds two atoms of Zn and is likely involved in protein-protein interactions. Many ring domain proteins play a role in the protein degradation pathway and E3 ubiquitin-protein ligase activity is thought to be a general function of this domain (Lorick et al. (1999) Proc Natl Acad Soc USA 96:11364-11369). The C3HC4 domain is also present in some transcription factors where it may be involved in protein interaction or regulation (Hakli et al. (2004)FEBS Lett 560:56-62). Since regulation of protein turnover/degradation is a key regulatory step in many biological processes (Hellmann and Estelle (2002) Science 297:793-797), misexpression of Clone 560731 may influence the turnover of proteins that are involved in nitrogen metabolism, thus conferring tolerance to low nitrate.

### EXAMPLE 4

Lead Summary: Lead 93-ME10822 (SEQ ID NO: 114)

| Construct | Event/Generation | Plant Stage | Assay | Result |
|---|---|---|---|---|
| 35S::At4g24700 | −01/T₂ segregating | Seedling | Low Nitrate | Significant at p ≦ .05 |
| | plants | | Tolerance | |
| 35S::At4g24700 | −02/T₂ segregating | Seedling | Low Nitrate | Significant at p ≦ .05 |
| | plants | | Tolerance | |
| 35S::At4g24700 | −03/T₂ segregating | Seedling | Low Nitrate | Significant at p ≦ .05 |
| | plants | | Tolerance | |
| 35S::At4g24700 | −01/T₃ segregating | Seedling | Low Nitrate | Significant at p ≦ .05 |
| | plants | | Tolerance | |
| 35S::At4g24700 | −02/T₃ segregating | Seedling | Low Nitrate | Significant at p ≦ .05 |
| | plants | | Tolerance | |
| 35S::At4g24700 | −03/T₃ segregating | Seedling | Low Nitrate | Significant at p ≦ .05 |
| | plants | | Tolerance | |

Ectopic expression of At4g24700 under the control of the 35S promoter induces the following phenotypes:

Enhanced growth after 14 days on low nitrate-containing media compared to controls.

ME10822 Was Identified from a Superpool Screen for Tolerance to Low Nitrate Conditions.

Superpools 72-81 were screened for seedlings that were larger or greener than controls on low nitrate growth media. For Superpools 72-81, transgene sequence was obtained for 24 candidate seedlings. One of the 24 candidate sequences aligned with ME10822 when analyzed with BLAST.

Two Events of ME10822 Show 3:1 Segregation for Finale® Resistance. One Event Shows 15:1 Segregation for Finale® Resistance.

Events −01 and −03 segregated 3:1 (R:S) for Finale® resistance in the T₂ generation and Event −02 segregated 15:1 (data not shown).

Three Events of ME10822 Showed Significantly Increased Growth under Low Nitrate Growth Conditions in Both Generations.

Seeds representing three events of ME10822 from each of the T₂ and the T₃ generations were sown as described in the Low Nitrate Assay. Both generations of events −01, −02 and −03 had the transgene linked to the enhanced growth phenotype at a confidence level of p <0.05 (Table 4-1).

TABLE 4-1
Chi-square comparison of enhanced growth between transgenic
seedlings and pooled non-transgenic segregants after 20 days
of growth on low nitrate (300 μM KNO₃ MS) media.

| | Enhanced | Arrested | | |
|---|---|---|---|---|
| Event | Growth | Growth | Chi-Square | p-value |
| ME10822-01 T₂ | 42 | 12 | 52.50 | 4.3E−13 |
| ME10822-01 CTRL | 0 | 36 | | |
| ME10822-01 T₃ | 47 | 16 | 51.13 | 8.64E−13 |
| ME10822-02 T₂ | 60 | 13 | 23.53 | 1.23E−06 |
| ME10822-03 CTRL | 1 | 9 | | |
| ME10822-02 T₃ | 49 | 24 | 11.98 | 5.37E−04 |
| ME10822-03 T₂ | 48 | 8 | 68.57 | 1.22E−16 |
| ME10822-03 CTRL | 0 | 40 | | |
| ME10822-03 T₃ | 50 | 9 | 68.49 | 1.28E−16 |

Qualitive Analysis of the T₁ Plants:

There were no observable differences in the physical appearance of the four T₁ plants was compared to the controls.

Qualitative and Quantitative Analysis of the T₂ Plants:

Events −01, −02, and −03 of ME10822 had leaves which appear slightly more oblong compared to controls.

At4g24700 encodes a 143 amino acid protein of unknown function. Microarray data (not shown) indicate that this sequence is positively regulated by light during the diurnal cycle. This sequence may be involved in photosynthetic related processes that could influence nitrogen metabolism and partitioning.

### EXAMPLE 5

Lead Summary: Lead 98-ME07523 (SEQ ID NO: 116)

| Construct | Event/Generation | Plant Stage | Assay | Result |
|---|---|---|---|---|
| 35S::14432 | −02/T₃ segregating plants | Seedling | Low Nitrate Tolerance | Significant at p ≦ .05 |
| 35S::14432 | −04/T₂ segregating plants | Seedling | Low Nitrate Tolerance | Significant at p ≦ .05 |
| 35S::14432 | −02/T₄ segregating plants | Seedling | Low Nitrate Tolerance | Significant at p ≦ .05 |
| 35S::14432 | −04/T₃ segregating plants | Seedling | Low Nitrate Tolerance | Significant at p ≦ .05 |

Ectopic expression of Clone 14432 under the control of the 35S promoter results in enhanced photosynthesis on low nitrate-containing media after 14 days compared to controls.

ME07523 Was Identified from a Superpool Screen for Seedling Tolerance to Low Nitrate Conditions.

Superpools 52-61 were screened for seedlings that were larger or greener than controls on low nitrate growth media (Ceres SOP 45-Low Nitrate Screen on Agar). For Superpools 52-61, transgene sequence was obtained for 23 candidate seedlings. Two of the 23 candidate sequences BLASTed to ME07523.

Two Events of ME07523 Show 3:1 Segregation for Finale® Resistance.

Events −02 and −04 segregated 3:1 (R:S) for Finale® resistance in the T₂ generation (data not shown).

Two Events of ME07523 Showed Significantly Increased Photosynthetic Efficiency under Low Nitrate Growth Conditions in Both Generations.

Two events of ME07523 were sown as described in the Low Nitrate Assay in both the T₂ and the T₃ generations (or T₃ and T₄ generations, as is the case for Event −02). In this study, the seedling photosynthetic efficiency was measured as Fv/Fm comparing transgenic plants within an event to non-transgenic segregants pooled across the same plate. Two events, −02 and −04, were significant in both generations at p=0.05, using a one-tailed t-test assuming unequal variance (Table 5-1).

TABLE 5-1
T-test comparison of seedling photosynthetic efficiency
between transgenic seedlings and pooled non-transgenic
segregants after 14 days of growth on low nitrate.

| | | | | Pooled Non- | | |
|---|---|---|---|---|---|---|
| | | Transgenic | | Transgenics | | t-test |
| Line | Events | Fv/Fm | n | Fv/Fm | n | p-value |
| ME07523 | ME07523-02 (T₃) | 0.62 | 29 | 0.58 | 25 | 2.1 × 10⁻³ |
| ME07523 | ME07523-02 (T₄) | 0.65 | 25 | 0.58 | 25 | 2.9 × 10⁻⁵ |
| ME07523 | ME07523-04 (T₂) | 0.62 | 28 | 0.55 | 24 | 1.6 × 10⁻³ |
| ME07523 | ME07523-04 (T₃) | 0.64 | 27 | 0.55 | 24 | 6.6 × 10⁻⁵ |

Qualitative Analysis of the T₁, Plants:

Event −03 appeared light green with a weak inflorescence. There were no observable differences in the physical appearance of all other events compared to wild-type.

Qualitative and Quantitative Analysis of the T₂ Plants:

There were no observable differences in the physical appearance of Events −02 and −04 of ME07523 compared to controls.

Clone 14432 encodes a 156 amino acid bZIP transcription factor with unknown function. bZIP transcription factors are know to regulate a wide variety of processes including light and stress signaling, seed maturation, flower development and pathogen defense (Jakoby et al. (2002) Trends Plant Sci 7:106-111). Mis-expression of a transcription factor that controls processes involving nitrogen and or carbon metabolism can condition tolerance to low nitrogen environments such as that observed for the Dof1 transcription factor (Yanagisawa et al., 2004).

### EXAMPLE 6

Lead Summary: Lead 112 - ME03926 (SEQ ID NO: 201)

| Construct | Event/Generation | Plant Stage | Assay | Result |
|---|---|---|---|---|
| 35S::150823 | −01/T₂ segregating plants | Seedling | Low Nitrate | Significant at p ≦ .05 |
| | | | Tolerance | |
| 35S::150823 | −03/T₂ segregating plants | Seedling | Low Nitrate | Significant at p ≦ .05 |
| | | | Tolerance | |
| 35S::150823 | −01/T₃ segregating plants | Seedling | Low Nitrate | Significant at p ≦ .05 |
| | | | Tolerance | |
| 35S::150823 | −03/T₃ segregating plants | Seedling | Low Nitrate | Significant at p ≦ .05 |
| | | | Tolerance | |

Ectopic expression of Clone 150823 under the control of the 35S promoter results in enhanced growth on low nitrate-containing media after 14 days compared to controls.

ME03926 Was Identified from a Superpool Screen for Seedling Tolerance to Low Nitrate Conditions.

Superpools 22-31 were screened for seedlings that were larger or greener than controls on low nitrate growth media (Ceres SOP 45-Low Nitrate Screen on Agar). For Superpools 22-31, transgene sequence was obtained for 40 candidate seedlings. Three of the 40 candidate sequences BLASTed to ME03926.

Two Events of ME03926 Segregate for a Single Insert.

Events −01 and −03 segregated 3:1 (R:S) for Finale® resistance in the T₂ generation (data not shown).

Two Events of ME03926 Showed Significantly Increased Growth under Low Nitrate Conditions in Both Generations.

Two events of ME03926 were sown as described in the Low Nitrate Assay with two slight differences, in both the T₂ and the T₃ generations. One difference was that 100 μM KNO³ media was used instead of the standard 300 μM KNO₃. The other difference was that 10 seeds were sown per plate instead of the standard 100 seeds. In this study, the qualitative growth of the plants was noted. A large portion of the plants continued to grow and flower on the plates after other plants were arrested in growth. A Chi-square comparison test was performed comparing transgenics to non-transgenic segregants (internal controls) with the enhanced growth versus arrested growth phenotypes to determine whether the increased growth was linked to the transgene. For both events, −01 and −03, in both generations, the transgene was linked to the enhanced growth phenotype with a confidence level of p<0.05 (Table 6-1).

TABLE 6-1
Chi-square comparison of enhanced growth between transgenic
seedlings and pooled non-transgenic segregants after 21 days of
growth on low nitrate (100 μM) media.

| Event | Enhanced Growth | Arrested Growth | Chi-Square | p-value |
|---|---|---|---|---|
| ME03926-01 T₂ | 12 | 30 | 11.87 | 5.71 × 10⁻⁴ |
| ME10822-01 CTRL | 1 | 44 | | |
| ME03926-01 T₃ | 11 | 28 | 11.52 | 6.9 × 10⁻⁴ |
| ME03926-03 T₂ | 13 | 22 | 16.63 | 4.55 × 10⁻⁴ |
| ME10822-03 CTRL | 1 | 44 | | |
| ME03926-03 T₃ | 9 | 27 | 9.59 | 1.96 × 10⁻³ |

Qualitative Analysis of the T₁, Plants:

There were no observable differences in the physical appearance of the four T₁, plants compared to the controls.

Qualitative and Quantitative Analyses of the T₂ Plants:

There was no observable difference in the physical appearance of Event −01 compared to the controls. Event −03 had a slightly smaller rosette and the leaves appeared slightly more oblong compared to controls.

Clone 150823 encodes a 516 amino acid glycosyl hydrolase family 9 protein. The immediate connection between glycosyl hydrolases and low nitrogen tolerance is not yet apparent. Additional work will be necessary to determine the mode of action for this gene and its effect on nitrogen utilization.

### EXAMPLE 7

Lead Summary: MEO7344 (SEQ ID NO: 140)

| Construct | Event/Generation | Plant Stage | Assay | Result |
|---|---|---|---|---|
| 35S::101255 | −02/T₂ segregating plants | Mature | Low N Tolerance on Soil | Significant at p ≦ .05 |
| 35S::101255 | −03/T₂ segregating plants | Mature | Low N Tolerance on Soil | Significant at p ≦ .05 |
| 35S::101255 | −02/T₃ segregating plants | Mature | Low N Tolerance on Soil | Significant at p ≦ .05 |
| 35S::101255 | −03/T₃ segregating plants | Mature | Low N Tolerance on Soil | Significant at p ≦ .05 |

Ectopic expression of Clone 10.1255 under the control of the 35S promoter induces the following phenotypes:

Enhanced photosynthesis efficiency on nitrogen-depleted soil 38 days after germination compared compared to controls.

ME07344 Was Identified from Superpool Screens for Seedling Tolerance to Low Nitrate and Low Ammonium Nitrate Conditions.

Superpools 52-61 and later 56-65 were screened for seedlings that were larger, greener, or had a higher photosynthetic efficiency than controls on low nitrate and low ammonium nitrate growth media. Eight of the 72 low nitrate tolerance candidates and one low ammonium nitrate candidate aligned to ME07344 when analyzed using BLAST.

Both Events of ME07344 Segregate for a Single Insert.

Events −02 and −03 segregated 3:1 (R:S) for Finale® resistance in the T₂ generation (data not shown).

Two Events of ME07344 Showed Significantly Enhanced Photosynthetic Efficiency under Low Nitrogen Conditions in Both Generations.

Two events of ME07344 were sown on Sunshine LP#5 soil in both the T₂ and the T₃ generations. In this study, the 4^{th} true leaf from each plant was collected on day 38 and analyzed on the CF imager for its Fv/Fm value. Transgenic plants within an event were compared to all non-transgenic plants, including the non-transgenic segregants and external controls. Events −02 and −03 were significant at p≦0.05, using a one-tailed t-test assuming unequal variance (Table 7.1).

TABLE 7.1
T-test comparison of photosynthetic efficiency between
transgenic plants and non-transgenic controls after 38 days
of growth on nitrogen-depleted soil.

| | | | | Non- | | |
|---|---|---|---|---|---|---|
| | | | | Transgenic | | |
| | | Transgenic | | Controls | | t-test |
| Line | Events | Fv/Fm | n | Fv/Fm | n | p-value |
| ME07344 | ME07344-02 (T₂) | 0.752 | 17 | 0.729 | 50 | 2.86 × 10⁻⁴ |
| ME07344 | ME07344-02 (T₃) | 0.750 | 16 | 0.729 | 50 | 1.11 × 10⁻⁴ |
| ME07344 | ME07344-03 (T₂) | 0.741 | 13 | 0.729 | 50 | 0.018 |
| ME07344 | ME07344-03 (T₃) | 0.754 | 17 | 0.729 | 50 | 4.62 × 10⁻⁶ |

Qualitative Analysis of the T₁, Plants:

Events −01 and −04 were dark green with oblong rosette leaves. Event −10 was dark green. The remaining events appeared wild-type.

Qualitative and Quantitative Analysis of the T₂ Plants:

There were no observable or statistical differences between events −02 and −03 of ME07344 and wild type plants for germination or fertility (as measured by silique number and seed fill).

Clone 101255 encodes a 359 amino acid CCCH-type zinc finger transcription factor from Arabidopsis. As described above, transcription factors may control expression of multiple genes in pathways and may ultimately affect a plant's nitrogen use efficiency and tolerance to low nitrogen growth conditions.

The results from the following Examples 8-10 confirm that the homologues to the above described Leads show improved NVE when assayed in the aboved-described assays.

### EXAMPLE 8

ME24939 SEQ ID NO: 200 (SEEDLING AREA AND PHOTOSYNTHETIC EFFICIENCY (P.E.))-Homolog of ME10822 (SEQ ID NO:201)

TABLE 8.1
T-test comparison of seedling area between transgenic seedlings
and pooled non-transgenic segregants across the same line after
14 days of growth on low nitrate.

| | | | | Pooled Non- | | |
|---|---|---|---|---|---|---|
| | | Transgenic | | Transgenics | | t-test |
| Line | Events | Area | n | Area | n | p-value |
| ME24939 | ME24939-01 (T₂) | 0.05239 | 17 | 0.04744 | 76 | 3.03 × 10⁻² |
| ME24939 | ME24939-03 (T₂) | 0.05663 | 15 | 0.04744 | 76 | 4.22 × 10⁻⁴ |
| ME24939 | ME24939-04 (T₂) | 0.07203 | 15 | 0.04744 | 76 | 9.91 × 10⁻⁸ |
| ME24939 | ME24939-09 (T₂) | 0.05713 | 16 | 0.04744 | 76 | 6.87 × 10⁻⁵ |
| ME24939 | ME24939-13 (T₂) | 0.05046 | 19 | 0.04744 | 76 | 4.90 × 10⁻² |
| ME24939 | ME24939-14 (T₂) | 0.05137 | 17 | 0.04744 | 76 | 1.40 × 10⁻² |
| ME24939 | ME24939-15 (T₂) | 0.06628 | 12 | 0.04744 | 76 | 1.14 × 10⁻⁵ |
| ME24939 | ME24939-17 (T₂) | 0.05480 | 20 | 0.04744 | 76 | 3.72 × 10⁻³ |
| ME24939 | ME24939-19 (T₂) | 0.05666 | 20 | 0.04744 | 76 | 1.46 × 10⁻³ |
| ME24939 | ME24939-20 (T₂) | 0.05262 | 17 | 0.04744 | 76 | 3.89 × 10⁻² |

TABLE 8.2
T-test comparison of seedling photosynthetic efficiency
between transgenic seedlings and pooled non-transgenic
segregants across the same line after 14 days of growth
on low nitrate.

| | | | | Pooled Non- | | |
|---|---|---|---|---|---|---|
| | | Transgenic | | Transgenics | | t-test |
| Line | Events | Fv/Fm | n | Fv/Fm | n | p-value |
| ME24939 | ME24939-01 (T₂) | 0.5959 | 17 | 0.5589 | 76 | 1.21 × 10⁻² |
| ME24939 | ME24939-08 (T₂) | 0.6150 | 9 | 0.5589 | 76 | 8.47 × 10⁻⁴ |
| ME24939 | ME24939-12 (T₂) | 0.6192 | 13 | 0.5589 | 76 | 4.96 × 10⁻⁶ |
| ME24939 | ME24939-13 (T₂) | 0.6163 | 19 | 0.5589 | 76 | 1.39 × 10⁻⁵ |
| ME24939 | ME24939-14 (T₂) | 0.5836 | 17 | 0.5589 | 76 | 4.75 × 10⁻² |
| ME24939 | ME24939-16 (T₂) | 0.6049 | 20 | 0.5589 | 76 | 1.05 × 10⁻³ |
| ME24939 | ME24939-17 (T₂) | 0.6491 | 20 | 0.5589 | 76 | 3.31 × 10⁻¹² |
| ME24939 | ME24939-18 (T₂) | 0.6040 | 18 | 0.5589 | 76 | 6.89 × 10⁻⁴ |
| ME24939 | ME24939-19 (T₂) | 0.6027 | 20 | 0.5589 | 76 | 2.03 × 10⁻³ |
| ME24939 | ME24939-20 (T₂) | 0.5896 | 17 | 0.5589 | 76 | 1.78 × 10⁻² |

### EXAMPLE 9

ME02730 (SEQ ID NO: 112) (P.E. ONLY)-Homolog of ME08309 (SEQ ID NO:107)

TABLE 9.1
T-test comparison of seedling photosynthetic efficiency
between transgenic seedlings and pooled non-transgenic
segregants across the same line after 14 days of growth
on low nitrate.

| | | | | Pooled Non- | | |
|---|---|---|---|---|---|---|
| | | Transgenic | | Transgenics | | t-test |
| Line | Events | Fv/Fm | n | Fv/Fm | n | p-value |
| ME02730 | ME02730-02 (T₂) | 0.6665 | 16 | 0.5996 | 38 | 8.95 × 10⁻⁵ |
| ME02730 | ME02730-03 (T₂) | 0.6797 | 19 | 0.5996 | 38 | 4.39 × 10⁻⁶ |
| ME02730 | ME02730-04 (T₂) | 0.6244 | 15 | 0.5996 | 38 | 8.73 × 10⁻⁴ |
| ME02730 | ME02730-05 (T₃) | 0.6985 | 11 | 0.5996 | 38 | 1.18 × 10⁻⁷ |

### EXAMPLE 10

ME05213 (P.E. AND SEEDLING DATA FOR ONE EVENT) SEQ ID NO:84-Homolog of ME02507 (SEQ ID NO:81)

TABLE 10.1
T-test comparison of seedling photosynthetic efficiency
between transgenic seedlings and pooled non-transgenic
segregants across the same plate after 14 days of growth
on low nitrate.

| | | | | Pooled Non- | | |
|---|---|---|---|---|---|---|
| | | Transgenic | | Transgenics | | t-test |
| Line | Events | Fv/Fm | n | Fv/Fm | n | p-value |
| ME05213 | ME05213-04 (T₂) | 0.6633 | 15 | 0.6161 | 22 | 2.79 × 10⁻⁵ |
| ME05213 | ME05213-05 (T₂) | 0.6772 | 14 | 0.6161 | 22 | 1.11 × 10⁻⁶ |

TABLE 10.2
T-test comparison of seedling area between transgenic seedlings
and pooled non-transgenic segregants across the same line after
14 days of growth on low nitrate.

| | | | | Pooled Non- | | |
|---|---|---|---|---|---|---|
| | | Transgenic | | Transgenics | | t-test |
| Line | Events | Area | n | Area | n | p-value |
| ME05213 | ME05213-03 (T₂) | 0.06102 | 16 | 0.05407 | 20 | 1.81 × 10⁻² |

### EXAMPLE 11

Determination of Functional Homolog Sequences

The “Lead” sequences described in above Examples are utilized to identify functional homologs of the lead sequences and, together with those sequences, are utilized to determine a consensus sequence for a given group of lead and functional homolog sequences.

A subject sequence is considered a functional homolog of a query sequence if the subject and query sequences encode proteins having a similar function and/or activity. A process known as Reciprocal BLAST (Rivera et al, (1998) Proc.Natl Acad. Sci. USA 95:6239-6244) is used to identify potential functional homolog sequences from databases consisting of all available public and proprietary peptide sequences, including NR from NCBI and peptide translations from Ceres clones.

Before starting a Reciprocal BLAST process, a specific query polypeptide is searched against all peptides from its source species using BLAST in order to identify polypeptides having sequence identity of 80% or greater to the query polypeptide and an alignment length of 85% or greater along the shorter sequence in the alignment. The query polypeptide and any of the aforementioned identified polypeptides are designated as a cluster.

The BLASTP version 2.0 program from Washington University at Saint Louis, Missouri, USA was used to determine BLAST sequence identity and E-value. The BLASTP version 2.0 program includes the following parameters: 1) an E-value cutoff of 1.0e-5; 2) a word size of 5; and 3) the -postsw option. The BLAST sequence identity was calculated based on the alignment of the first BLAST HSP (High-scoring Segment Pairs) of the identified potential functional homolog and/or ortholog sequence with a specific query polypeptide. The number of identically matched residues in the BLAST HSP alignment was divided by the HSP length, and then multiplied by 100 to get the BLAST sequence identity. The HSP length typically included gaps in the alignment, but in some cases gaps can be excluded.

The main Reciprocal BLAST process consists of two rounds of BLAST searches; forward search and reverse search. In the forward search step, a query polypeptide sequence, “polypeptide A,” from source species S^{A} is BLASTed against all protein sequences from a species of interest. Top hits are determined using an E-value cutoff of 10⁻⁵ and an identity cutoff of 35%. Among the top hits, the sequence having the lowest E-value is designated as the best hit, and considered a potential functional homolog. Any other top hit that had a sequence identity of 80% or greater to the best hit or to the original query polypeptide is considered a potential functional homolog as well. This process is repeated for all species of interest.

In the reverse search round, the top hits identified in the forward search from all species are used to perform a BLAST search against all protein or polypeptide sequences from the source species S^{A}. A top hit from the forward search that returned a polypeptide from the aforementioned cluster as its best hit is also considered as a potential functional homolog.

Functional homologs are identified by manual inspection of potential functional homolog sequences. Representative functional homologs are shown in FIGS. 1-5. The Figures represents a grouping of a lead/query sequence aligned with the corresponding identified functional homolog subject sequences. Lead sequences and their corresponding functional homolog sequences are aligned to identify conserved amino acids and to determine a consensus sequence that contains a frequently occurring amino acid residue at particular positions in the aligned sequences, as shown in FIGS. 1-5.

Each consensus sequence then is comprised of the identified and numbered conserved regions or domains, with some of the conserved regions being separated by one or more amino acid residues, represented by a dash (-), between conserved regions.

Useful polypeptides of the inventions, therefore, include each of the lead and functional homolog sequences shown in FIGS. 1-5, as well as the consensus sequences shown in the Figures. The invention also encompasses other useful polypeptides constructed based upon the consensus sequence and the identified conserved regions. Thus, useful polypeptides include those which comprise one or more of the numbered conserved regions in each alignment table in FIGS. 1-5, wherein the conserved regions may be separated by dashes. Useful polypeptides also include those which comprise all of the numbered conserved regions in FIGS. 1-5, alternatively comprising all of the numbered conserved regions in an individual alignment table and in the order as depicted in FIGS. 1-5. Useful polypeptides also include those which comprise all of the numbered conserved regions in the alignment table and in the order as depicted in FIGS. 1-5, wherein the conserved regions are separated by dashes, wherein each dash between two adjacent conserved regions is comprised of the amino acids depicted in the alignment table for lead and/or functional homolog sequences at the positions which define the particular dash. Such dashes in the consensus sequence can be of a length ranging from length of the smallest number of dashes in one of the aligned sequences up to the length of the highest number of dashes in one of the aligned sequences.

Such useful polypeptides can also have a length (a total number of amino acid residues) equal to the length identified for a consensus sequence or of a length ranging from the shortest to the longest sequence in any given family of lead and functional homolog sequences identified in FIGS. 1-5.

The present invention further encompasses nucleotides that encode the above described polypeptides, as well as the complements thereof, and including alternatives thereof based upon the degeneracy of the genetic code.

The invention being thus described, it will be apparent to one of ordinary skill in the art that various modifications of the materials and methods for practicing the invention can be made. Such modifications are to be considered within the scope of the invention as defined by the following claims.

The following references are cited in the Specification. Each of the references from the patent and periodical literature cited herein is hereby expressly incorporated in its entirety by such citation.

### REFERENCES

(1) Zhang et al. (2004) Plant Physiol. 135:615.
(2) Salomon et al. (1984) EMBO J. 3:141.
(3) Herrera-Estrella et al. (1983) EMBO J. 2:987.
(4) Escudero et al. (1996) Plant J. 10:355.
(5) Ishida et al. (1996) Nature Biotechnology 14:745.
(6) May et al. (1995) Bio/Technology 13:486)
(7) Armaleo et al. (1990) Current Genetics 17:97.
(8) Smith. T.F. and Waterman, M.S. (1981) Adv. App. Math. 2:482.
(9) Needleman and Wunsch (1970) J. Mol. Biol. 48:443.
(10) Pearson and Lipman (1988) Proc. Natl. Acad. Sci. (USA) 85: 2444.
(11) Yamauchi et al. (1996) Plant Mol Biol. 30:321-9.
(12) Xu et al. (1995) Plant Mol. Biol. 27:237.
(13) Yamamoto etal. (1991) Plant Cell 3:371.
(14) P. Tijessen, “Hybridization with Nucleic Acid Probes” In Laboratory Techniques in Biochemistry and Molecular Biology, P.C. vand der Vliet, ed., c. 1993 by Elsevier, Amsterdam.
(15) Bonner et al., (1973) J. Mol. Biol. 81:123.
(16) Sambrook et al., Molecular Cloning: A Laboratory Manual, Second Edition, Cold Spring Harbor Laboratory Press, 1989, New York.
(17) Shizuya et al. (1992) Proc. Natl. Acad. Sci. USA, 89: 8794-8797.
(18) Hamilton et al. (1996) Proc. Natl. Acad. Sci. USA, 93: 9975-9979.
(19) Burke et al. (1987) Science, 236:806-812.
(20) Sternberg N. et al. (1990) Proc. Natl. Acad. Sci. USA., 87:103-7.
(21) Bradshaw et al. (1995) Nucl Acids Res, 23: 4850-4856.
(22) Frischauf et al. (1983) J. Mol. Biol., 170: 827-842.
(23) Huynh et al., Glover NM (ed) DNA Cloning: A practical Approach, Vol.1 Oxford: IRL Press (1985).
(24) Walden et al. (1990) Mol Cell Biol 1: 175-194.
(25) Vissenberg et al. (2005) Plant Cell Physiol 46:192.
(26) Husebye et al. (2002) Plant Physiol 128:1180.
(27) Plesch et al. (2001) Plant J. 28:455.
(28) Weising et al. (1988) Ann. Rev. Genet., 22:421.
(29) Christou (1995) Euphytica, v. 85, n.1-3:13-27.
(30) Newell (2000)
(31) Griesbach (1987) Plant Sci. 50:69-77.
(32) Fromm et al. (1985) Proc. Natl. Acad. Sci. USA 82:5824.
(33) Paszkowski et al. (1984) EMBO J. 3:2717.
(34) Klein et al. (1987) Nature 327:773.
(35) Willmitzer, L. (1993) Transgenic Plants. In: iotechnology, A Multi-Volume Comprehensive treatise (H. J. Rehm, G. Reed, A. Puler, P. Stadler, eds., Vol. 2, 627-659, VCH Weinheim-New York-Basel-Cambridge).
(36) Crit. Rev. Plant. Sci. 4:1-46.
(37) Fromm et al. (1990) Biotechnology 8:833-844.
(38) Cho et al. (2000) Planta 210:195-204.
(39) Brootghaerts et al. (2005) Nature 433:629-633.
(40) Lincoln et al. (1998) Plant Mol. Biol. Rep. 16:1-4.
(41) Lacomme et al. (2001), “Genetically Engineered Viruses” (C. J. A. Ring and E. D. Blair, Eds). Pp. 59-99, BIOS Scientific Publishers, Ltd. Oxford, UK.
(42) Good, A. G., Shrawat, A. K., and Muench, D. G. (2004). Can less yield more? Is reducing nutrient input into the environment compatible with maintaining crop production? Trends Plant Sci 9, 597-605.
(43) Hakli, M., Lorick, K. L., Weissman, A. M., Janne, O. A., and Palvimo, J. J. (2004).

Transcriptional coregulator SNURF (RNF4) possesses ubiquitin E3 ligase activity.

FEBS Lett 560, 56-62.
(44) Hellmann, H., and Estelle, M. (2002). Plant development: regulation by protein degradation. Science 297, 793-797.
(45) Hernandez, J. M., Heine, G. F., Irani, N. G., Feller, A., Kim, M. G., Matulnik, T., Chandler, V. L., and Grotewold, E. (2004). Different mechanisms participate in the R-dependent activity of the R2R3 MYB transcription factor C1. J Biol Chem 279, 48205-48213.
(46) Jakoby, M., Weisshaar, B., Droge-Laser, W., Vicente-Carbajosa, J., Tiedemann, J., Kroj, T., and Parcy, F. (2002). bZIP transcription factors in Arabidopsis. Trends Plant Sci 7, 106-111.
(47) Lorick, K. L., Jensen, J. P., Fang, S., Ong, A. M., Hatakeyama, S., and Weissman, A. M. (1999). RING fingers mediate ubiquitin-conjugating enzyme (E2)-dependent ubiquitination. Proc Natl Acad Sci U S A 96, 11364-11369
(48) Marker, C., Zemann, A., Terhorst, T., Kiefmann, M., Kastenmayer, J. P., Green, P., Bachellerie, J. P., Brosius, J., and Huttenhofer, A. (2002). Experimental RNomics: identification of 140 candidates for small non-messenger RNAs in the plant Arabidopsis thaliana. Curr Biol 12, 2002-2013.
(49) Sainz, M. B., Grotewold, E., and Chandler, V. L. (1997). Evidence for direct activation of an anthocyanin promoter by the maize C1 protein and comparison of DNA binding by related Myb domain proteins. Plant Cell 9, 611-625.
(50) Tang, T. H., Polacek, N., Zywicki, M., Huber, H., Brugger, K., Garrett, R., Bachellerie, J. P., and Huttenhofer, A. (2005). Identification of novel non-coding RNAs as potential antisense regulators in the archaeon Sulfolobus solfataricus. Mol Microbiol 55, 469-481.
(51) Todd, C. D., Zeng, P., Huete, A. M., Hoyos, M. E., and Polacco, J. C. (2004). Transcripts of MYB-like genes respond to phosphorous and nitrogen deprivation in Arabidopsis. Planta 219, 1003-1009.
(52) Yanagisawa, S., Akiyama, A., Kisaka, H., Uchimiya, H., and Miwa, T. (2004).

Metabolic engineering with Dofl transcription factor in plants: Improved nitrogen assimilation and growth under low-nitrogen conditions. Proc Natl Acad Sci U S A 101, 7833-7838.
(53) Derlot et al. (2001) Amino Acid Transport. In Plant Nitrogen (eds. Lea and Morot-Gaudry), pp. 167-212. Springer-Verlag, Berlin, Heidelberg
(54) Glass et al. (2002) J. Exp. Bot. 53: 855-864
(55) Krapp et al. (2002) Nitrogen and Signaling. In Photosynthetic Nitrogen Assimilation and Associated Carbon Respiratory Metabolism (eds. Foyer and Noctor), pp. 205-225. Kluwer Academic Publisher, Dordrecht, The Netherlands
(56) Touraine et al. (2001) Nitrate uptake and its regulation. In Plant Nitrogen (eds. Lea and Morot-Gaudry), pp. 1-36. Springer-Verlag, Berlin, Heidelberg.
(57) Redinbaugh, M. G., et al. (1991) Physiol. Plant. 82, 640-650.
(58) Huber, J. L., et al. (1994) Plant Physiol 106, 1667-1674.
(59) Hwang, C. F., et al. (1997) Plant Physiol. 113, 853-862.
(60) Redinbaugh, M. G., et al. (1998) Plant Science 134, 129-140.
(61) Gazzarrini, S., et al. (1999) Plant Cell 11, 937-948.
(62) Glass, A. D. M., et al. (2002) J. Exp. Bot. 53, 855-864.
(63) Okamoto, M., et al. (2003) Plant Cell Physiol. 44, 304-317.
(64) Rastogi, R., et al. (1993) Plant J. 4, 317-326.
(65) Lin, Y., et al. (1994) Plant Physiol. 106, 477-484.
(66) Wang, R., et al. (2000) Plant Cell 12, 1491-1510.
(67) Wang, R., et al. (2003) Plant Physiol. 132, 556-567
(68) Forde, B. G. (2002) Annual Review of Plant Biology 53, 203-224
(69) Yamaya, T., Obara, M., Nakajima, H., Sasaki, S., Hayakawa, T., and Sato, T. (2002). Genetic manipulation and quantitative-trait loci mapping for nitrogen recycling in rice. J. Exp. Bot. 53, 917-925

What is claimed is:

1. A method of improving nitrogen use efficiency, modulating vegetative growth, seedling vigor and/or plant biomass, said method comprising introducing into a plant cell an isolated nucleic acid comprising a nucleotide sequence selected from the group consisting of:
(a) a nucleotide sequence that encodes an amino acid sequence that is at least 85% identical to any one of Leads 82, 85, 92, 93, 98, 112, ME07344, ME05213, ME02730 and ME24939, SEQ ID NOs: 81, 105, 107, 114, 116, 201, 140, 84, 112 and 200, respectively;
(b) a nucleotide sequence that is complementary to any one of the nucleotide sequences according to paragraph (a);
(c) a nucleotide sequence according to any one of SEQ ID NOs: 80, 104, 106, 113, 115, 127, 139, 202, 203 and 204;
(d) a nucleotide sequence that is an interfering RNA to the nucleotide sequence according to paragraph (a);
(e) a nucleotide sequence able to form a hybridized nucleic acid duplex with the nucleic acid according to any one of paragraphs (a)-(d) at a temperature from about 40° C. to about 48° C. below a melting temperature of the hybridized nucleic acid duplex;
(f) a nucleotide sequence encoding any one of the amino acid sequences identified as Leads 82, 85, 92, 93, 98, 112, ME07344, ME05213, ME02730 and ME24935, corresponding to SEQ ID NOs: 81, 105, 107, 114, 116, 201, 140, 84, 112 and 200, respectively; or
(g) a nucleotide sequence encoding any one of the lead, functional homolog or consensus sequences in FIGS. 1-5, wherein said plant produced from said plant cell has improved nitrogen use efficiency, modulated plant size, modulated vegetative growth, modulated seeding vigor and/or modulated biomass as compared to the corresponding level in tissue of a control plant that does not comprise said nucleic acid.

2. The method according to claim 1, wherein said consensus sequence comprises one or more of the conserved regions identified in any one of the alignment tables in FIGS. 1-5.

3. The method according to claim 2, wherein said consensus sequence comprises all of the conserved regions identified in the alignment tables in FIGS. 1-5.

4. The method according to claim 3, wherein said consensus sequence comprises all of the conserved regions and in the order identified in the alignment tables in FIGS. 1-5.

5. The method according to claim 4, wherein said conserved regions are separated by one or more amino acid residues.

6. The method according to claim 5, wherein each of said of one or more amino acids consisting in number and kind of the amino acids depicted in the alignment table for the lead and/or functional homolog sequences at the corresponding positions that define that gap.

7. The method according to claim 6, wherein said consensus sequence has a length in terms of total number of amino acids that is equal to the length identified for a consensus sequence in FIGS. 1-5, or equal to a length ranging from the shortest to the longest sequence in FIGS. 1-5.

8. The method of claim 1, wherein said difference is an increase in the level of nitrogen use efficiency, plant size, vegetative growth, seedling vigor and/or biomass.

9. The method of claim 1, wherein said isolated nucleic acid is operably linked to a regulatory region.

10. The method of claim 9, wherein said regulatory region is a promoter selected from the group consisting of YP0092 (SEQ ID NO: 38), PT0676 (SEQ ID NO: 12), PT0708 (SEQ ID NO: 17), PT0613 (SEQ ID NO: 5), PT0672 (SEQ ID NO: 11), PT0678 (SEQ ID NO: 13), PT0688 (SEQ ID NO: 15), PT0837 (SEQ ID NO: 24), the napin promoter, the Arcelin-5 promoter, the phaseolin gene promoter, the soybean trypsin inhibitor promoter, the ACP promoter, the stearoyl-ACP desaturase gene, the soybean α′ subunit of β-conglycinin promoter, the oleosin promoter, the 15 kD zein promoter, the 16 kD zein promoter, the 19 kD zein promoter, the 22 kD zein promoter, the 27 kD zein promoter, the Osgt-1 promoter, the beta-amylase gene promoter, the barley hordein gene promoter, p326 (SEQ ID NO: 76), YP0144 (SEQ ID NO: 55), YP0190 (SEQ ID NO: 59), p13879 (SEQ ID NO: 75), YP0050 (SEQ ID NO: 35), p32449 (SEQ ID NO: 77), 21876 (SEQ ID NO: 1), YP0158 (SEQ ID NO: 57), YP0214 (SEQ ID NO: 61), YP0380 (SEQ ID NO: 70), PT0848 (SEQ ID NO: 26), and PT0633 (SEQ ID NO:7), the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, ubiquitin promoters such as the maize ubiquitin-1 promoter, ribulose-1,5-bisphosphate carboxylase (RbcS) promoters such as the RbcS promoter from eastern larch (Larix laricina), the pine cab6 promoter, the Cab-1 gene promoter from wheat, the CAB-1 promoter from spinach, the cab1R promoter from rice ,the pyruvate orthophosphate dikinase (PPDK) promoter from corn, the tobacco Lhcb1*2 promoter, the Arabidopsis thaliana SUC2 sucrose-H+symporter promoter, and thylakoid membrane protein promoters from spinach (psaD, psaF, psaE, PC, FNR, atpC, atpD, cab, rbcS, PT0535 (SEQ ID NO: 3), PT0668 (SEQ ID NO: 2), PT0886 (SEQ ID NO: 29), PR0924 (SEQ ID NO: 78), YP0144 (SEQ ID NO: 55), YP0380 (SEQ ID NO: 70) and PT0585 (SEQ ID NO: 4).

11. A plant cell comprising an isolated nucleic acid comprising a nucleotide sequence selected from the group consisting of:
(a) a nucleotide sequence encoding an amino acid sequence that is at least 85% identical to any one of Leads 82, 85, 92, 93, 98, 112, ME07344, ME05213, ME02730 and ME24939, corresponding to SEQ ID NOs: 81, 105, 107, 114, 116, 201, 140, 84, 112 and 120, respectively;
(b) a nucleotide sequence that is complementary to any one of the nucleotide sequences according to paragraph (a);
(c) a nucleotide sequence according to any one of SEQ ID NOs: 80, 104, 106, 113, 115, 127, 139, 202, 203 and 204;
(d) a nucleotide sequence that is an interfering RNA to the nucleotide sequence according to paragraph (a);
(e) a nucleotide sequence able to form a hybridized nucleic acid duplex with the nucleic acid according to any one of paragraphs (a)-(c) at a temperature from about 40° C. to about 48° C. below a melting temperature of the hybridized nucleic acid duplex;
(f) a nucleotide sequence encoding any one of the amino acid sequences identified as Leads 82, 85, 92, 93, 98, 112, ME07344, ME05213, ME02730 and ME24935, corresponding to SEQ ID NOs: 81, 105, 107, 114, 116, 201, 140, 84, 112 and 200, respectively; or
(g) a nucleotide sequence encoding any one of the lead, functional homolog or consensus sequences in FIGS. 1-5.

12. A transgenic plant comprising the plant cell of claim 11.

13. Progeny of the plant of claim 12, wherein said progeny has modulated plant size, modulated vegetative growth, modulated plant architecture, modulated seedling vigor and/or modulated biomass as compared to the corresponding level in tissue of a control plant that does not comprise said nucleic acid.

14. Progeny of the plant of claim 12, wherein said progeny has improved nitrogen use efficiently as compared to a control plant that does not comprise said nucleic acid.

15. Seed from a transgenic plant according to claim 12.

16. Vegetative tissue from a transgenic plant according to claim 12.

17. A food product comprising vegetative tissue from a transgenic plant according to claim 12.

18. A feed product comprising vegetative tissue from a transgenic plant according to claim 12.

19. A product comprising vegetative tissue from a transgenic plant according to claim 12 used for the conversion into fuel or chemical feedstocks.

20. A method for improving nitrogen use efficiency and/or the biomass of a plant, said method comprising altering the level of expression in said plant of a nucleic acid molecule comprising a nucleotide sequence selected from the group consisting of:
(a) a nucleotide sequence that encodes an amino acid sequence that is at least 85% identical to any one of Leads 82, 85, 92, 93, 98, 112, ME07344, ME05213, ME02730 and ME24939, SEQ ID NOs: 81, 105, 107, 114, 116, 201, 140, 84, 112 and 200, respectively;
(b) a nucleotide sequence that is complementary to any one of the nucleotide sequences according to paragraph (a);
(c) a nucleotide sequence according to any one of SEQ ID NOs: 80, 104, 106, 113, 115, 127, 139,202,203and204;
(d) a nucleotide sequence that is an interfering RNA to the nucleotide sequence according to paragraph (a);
(e) a nucleotide sequence able to form a hybridized nucleic acid duplex with the nucleic acid according to any one of paragraphs (a)-(d) at a temperature from about 40° C. to about 48° C. below a melting temperature of the hybridized nucleic acid duplex;
(f) a nucleotide sequence encoding any one of the amino acid sequences identified as Leads 82, 85, 92, 93, 98, 112, ME07344, ME05213, ME02730 and ME24935, corresponding to SEQ ID NOs: 81, 105, 107, 114, 116, 201, 140, 84, 112 and 200, respectively; or
(g) a nucleotide sequence encoding any one of the lead, functional homolog or consensus sequences in FIGS. 1-5, wherein said plant produced from said plant cell has improved nitrogen use efficiency, modulated plant size, modulated vegetative growth, modulated seeding vigor and/or modulated biomass as compared to the corresponding level in tissue of a control plant that does not comprise said nucleic acid.

21. A method for detecting a nucleic acid in a sample, comprising: providing an isolated nucleic acid according to claim 1;
contacting said isolated nucleic acid with a sample under conditions that permit a comparison of the nucleotide sequence of the isolated nucleic acid with a nucleotide sequence of nucleic acid in the sample; and
analyzing the comparison.

22. A method for promoting improved nitrogen use efficiency and/or increased biomass in a plant, comprising:
(a) transforming a plant with a nucleic acid molecule comprising a nucleotide sequence encoding any one of the lead, functional homolog or consensus sequences in FIGS. 1-5; and
(b) expressing said nucleotide sequence in said transformed plant, whereby said transformed plant has an increased nitrogen use efficiency and/or biomass or enhanced seedling vigor as compared to a plant that has not been transformed with said nucleotide sequence.

23. An isolated nucleic acid molecule comprising:
(a) a nucleotide sequence that encodes an amino acid sequence that is at least 85% identical to any one of Leads 82, 85, 92, 93, 98, 112, ME07344, ME05213, ME02730 and ME24939, SEQ ID NOs: 81, 105, 107, 114, 116, 201, 140, 84, 112 and 200, respectively;
(b) a nucleotide sequence that is complementary to any one of the nucleotide sequences according to paragraph (a);
(c) a nucleotide sequence according to any one of SEQ ID NOs: 80, 104, 106, 113, 115, 127, 139,202,203and204;
(d) a nucleotide sequence that is an interfering RNA to the nucleotide sequence according to paragraph (a);
(e) a nucleotide sequence able to form a hybridized nucleic acid duplex with the nucleic acid according to any one of paragraphs (a)-(c) at a temperature from about 40° C. to about 48° C. below a melting temperature of the hybridized nucleic acid duplex;
(f) a nucleotide sequence encoding any one of the amino acid sequences identified as Leads 82, 85, 92, 93, 98, 112, ME07344, ME05213, ME02730 and ME24935, corresponding to SEQ ID NOs: 81, 105, 107, 114, 116, 201, 140, 84, 112 and 200, respectively; or
(g) a nucleotide sequence encoding any one of the lead, functional homolog or consensus sequences in FIGS. 1-5.

24. A vector, comprising:
a) a first nucleic acid having a regulatory region encoding a plant transcription and/or translation signal; and
b) a second nucleic acid having a nucleotide sequence according to any one of the nucleotide sequences of claim 23, wherein said first and second nucleic acids are operably linked.
